(19) United States
(12) Patent Application Publication
(10) Pub. No.: US 2025/0064873 A1
(43) Pub. Date: Feb. 27, 2025

(54) TNFSF-L Fusion Proteins and Uses Thereof

(71) Applicant: KaliVir Immunotherapeutics, Inc., Pittsburgh, PA (US)

(72) Inventors: Stephen Howard THORNE, Pittsburgh, PA (US); Mingrui ZHANG, Pittsburgh, PA (US)

(21) Appl. No.: 18/536,948

(22) Filed: Dec. 12, 2023

Related U.S. Application Data
(63) Continuation of application No. PCT/US2022/033524, filed on Jun. 15, 2022.
(60) Provisional application No. 63/211,766, filed on Jun. 17, 2021.

(51) Int. Cl. A61K 35/768; A61K 38/17; A61K 38/19; A61P 35/00; C12N 7/00

(52) U.S. Cl. CPC A61K 35/768; A61K 38/1732; A61K 38/191; A61P 35/00; C12N 7/00; C12N 2710/24122; C12N 2710/24132

(57) ABSTRACT

The present disclosure provides for compositions comprising nucleic acids encoding for fusion proteins of the Tumor Necrosis Factor Super Family Ligands fused to oligomerization domains. Further provided are viral and non-viral vectors for delivery of such compositions. The present disclosure also provides for compositions comprising fusion proteins of the Tumor Necrosis Factor Super Family Ligands fused to oligomerization domains.

### CROSS-REFERENCE

This application claims the benefit of PCT/US2022/033524 filed Jun. 15, 2022, which claims the benefit of U.S. Provisional Application No. 63/211,766 filed Jun. 17, 2021, which is incorporated by reference herein in its entirety.

### SEQUENCE LISTING

The instant application contains a Sequence Listing which has been submitted electronically in XML format and is hereby incorporated by reference in its entirety. Said XML copy, created on Nov. 14, 2023, is named 199249-715301_SL.XML and is 49,152 bytes in size.

### BACKGROUND

The TNF superfamily (TNFSF) is made up of proteins important for the development and functioning of many mammalian systems, including the immune, hematological, and skeletal systems. TNFSF proteins are ligands for a corresponding set of receptors of the TNF receptor superfamily (TNFS). TNFSF members are generally expressed as Type II membrane proteins, with the exception of lymphotoxin-alpha which is produced as a secreted protein.

To produce soluble forms of TNFSF proteins, generally, the membrane protein is often expressed in a cell line possessing a protease capable of separating the TNFSF extracellular domain from the transmembrane domain or a truncated form of the TNFSF protein is produced which has the extracellular domain plus a signal sequence. In either case, certain soluble forms of TNFSF ligand (TNFSF-L) proteins are often unstable in solution as homotrimers composed solely of the extracellular domain. Thus, there is a need for more stable forms of these important signaling proteins.

### BRIEF SUMMARY

Provided herein are compositions, wherein the composition comprises a nucleic acid, wherein the nucleic acid encodes for a fusion protein, wherein the fusion protein comprises: a TNF-superfamily ligand (TNFSF-L) or functional variant thereof; and a plurality of domains from a collectin family protein, wherein the plurality of domains from the collectin family protein comprises: an oligomerization domain or functional variant thereof; and a neck domain or functional variant thereof, wherein the oligomerization domain or functional variant thereof and the neck domain or functional variant thereof are closer in sequence proximity compared to their location in the collectin family protein. Further provided are compositions, wherein the fusion protein comprises, in N-terminal to C-terminal order: the oligomerization domain or functional variant thereof; the neck domain or functional variant thereof; optionally, a linker sequence; and the TNFSF-L or functional variant thereof. Further provided are compositions, wherein the TNFSF-L comprises a Lymphotoxin alpha, OX40 ligand, CD40 ligand, Fas ligand, CD27 ligand, CD30 ligand, 4-1BBL, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. Further provided are compositions, wherein the TNFSF-L is a CD40 ligand. Further provided are compositions, wherein the CD40 ligand comprises a sequence comprising at least 85% sequence identity to SEQ ID NO: 1. Further provided are compositions, wherein the TNFSF-L is an OX40 ligand. Further provided are compositions, wherein the OX40 ligand comprises a sequence comprising at least 85% sequence identity to SEQ ID NO: 2 or SEQ ID NO: 3. Further provided are compositions, wherein the TNFSF-L is a 4-1BBL. Further provided are compositions, wherein the 4-1BBL comprises a sequence comprising at least 85% sequence identity to SEQ ID NO: 4 or SEQ ID NO: 5. Further provided are compositions, wherein the TNFSF-L is a LIGHT. Further provided are compositions, wherein the LIGHT comprises a sequence comprising at least 85% sequence identity to SEQ ID NO: 6. Further provided are compositions, wherein the TNFSF-L is a TNF superfamily member 18 ligand (GITRL). Further provided are compositions, wherein the GITRL comprises a sequence comprising at least 85% sequence identity to SEQ ID NO: 7 or SEQ ID NO: 8. Further provided are compositions, wherein the collectin family protein is SP-A, SP-D, mannose binding lectin (MBL), conglutinin, CL-43, CL-L1, CL-K1, CL-P1, or CL-46. Further provided are compositions, wherein the collectin family protein is SP-D. Further provided are compositions, wherein the oligomerization domain comprises a sequence comprising at least 85% sequence identity to SEQ ID NO: 9. Further provided are compositions, wherein the oligomerization domain and the neck domain together comprise a sequence comprising at least 85% sequence identity to SEQ ID NO: 10. Further provided are compositions, wherein the oligomerization domain and the neck domain together comprise SEQ ID NO: 10. Further provided are compositions, wherein the fusion protein comprises the linker sequence, and wherein the linker sequence comprises GSG (glycine-serine-glycine) (SEQ ID NO: 12). Further provided are compositions, wherein the nucleic acid comprises an RNA. Further provided are compositions, wherein the nucleic acid comprises a DNA.

Provided herein are compositions, wherein the composition comprises: a nucleic acid, wherein the nucleic acid comprises a sequence comprising at least 85% sequence identity to SEQ ID NOS: 21, 22, 23, 24, 25, 26, or 27.

Provided herein are compositions, wherein the composition comprises: a fusion protein, wherein the fusion protein comprises a sequence comprising at least 85% sequence identity to SEQ ID NOS: 14, 15, 16, 17, 18, 19, or 20.

Provided herein are compositions, wherein the composition comprises: a vector; and a nucleic acid encoding for a TNF-superfamily ligand (TNFSF-L) or functional variant thereof, wherein the TNFSF-L or functional variant thereof is fused to an oligomerization domain. Further provided herein are compositions, wherein the nucleic acid encodes for a fusion protein, wherein the fusion protein comprises: a TNF-superfamily ligand (TNFSF-L) or functional variant thereof; and a plurality of domains from a collectin family protein, wherein the plurality of domains from the collectin family protein comprises: an oligomerization domain or functional variant thereof; and a neck domain or functional variant thereof, wherein the oligomerization domain or functional variant thereof and the neck domain or functional variant thereof are closer in sequence proximity compared to their location in the collectin family protein. Further provided herein are compositions, wherein the vector is a viral vector or non-viral vector. Further provided herein are compositions, wherein the non-viral vector comprises a nanoparticle carrier. Further provided herein are compositions, wherein the non-viral vector comprises a lipid nanoparticle carrier. Further provided herein are compositions, wherein the nanoparticle carrier comprises gold, silica, carbon nanotubes, water soluble fullerenes, silicon nanowires, quantum dots, or any combination thereof. Further provided herein are compositions, wherein the vector comprises a bacteriophage, virus-like particles (VLP), erythrocyte ghosts, bactofection, exosomes, or any combination thereof. Further provided herein are compositions, wherein the fusion protein comprises, in N-terminal to C-terminal order: the oligomerization domain or functional variant thereof; the neck domain or functional variant thereof; optionally, a linker sequence; and the TNFSF-L or functional variant thereof. Further provided herein are compositions, wherein the TNFSF-L comprises Lymphotoxin alpha, OX40 ligand, CD40 ligand, Fas ligand, CD27 ligand, CD30 ligand, 4-1BBL, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. Further provided herein are compositions, wherein the TNFSF-L is a CD40 ligand. Further provided herein are compositions, wherein the CD40 ligand comprises a sequence comprising at least 85% sequence identity to SEQ ID NO: 1. Further provided herein are compositions, wherein the TNFSF-L is an OX40 ligand. Further provided herein are compositions, wherein the OX40 ligand comprises a sequence comprising at least 85% sequence identity to SEQ ID NO: 2 or SEQ ID NO: 3. Further provided herein are compositions, wherein the TNFSF-L is a 4-1BBL. Further provided herein are compositions, wherein the 4-1BBL comprises a sequence comprising at least 85% sequence identity to SEQ ID NO: 4 or SEQ ID NO: 5. Further provided herein are compositions, wherein the TNFSF-L is a LIGHT. Further provided herein are compositions, wherein the LIGHT comprises a sequence comprising at least 85% sequence identity to SEQ ID NO: 6. Further provided are compositions, wherein the TNFSF-L is a TNF superfamily member 18 ligand (GITRL). Further provided are compositions, wherein the GITRL comprises a sequence comprising at least 85% sequence identity to SEQ ID NO: 7 or SEQ ID NO: 8. Further provided herein are compositions, wherein the collectin family protein comprises SP-A, SP-D, mannose binding lectin (MBL), conglutinin, CL-43, CL-L1, CL-K1, CL-P1, or CL-46. Further provided herein are compositions, wherein the collectin family protein is SP-D. Further provided herein are compositions, wherein the oligomerization domain comprises a sequence comprising at least 85% sequence identity to SEQ ID NO: 9. Further provided herein are compositions, wherein the oligomerization domain and the neck domain together comprise a sequence comprising at least 85% sequence identity to SEQ ID NO: 10. Further provided herein are compositions, wherein the oligomerization domain and the neck domain together comprise SEQ ID NO: 10. Further provided herein are compositions, wherein the fusion protein comprises the linker sequence, and wherein the linker sequence comprises GSG (glycine-serine-glycine) (SEQ ID NO: 12). Further provided herein are compositions, wherein the nucleic acid comprises an RNA. Further provided herein are compositions, wherein the nucleic acid comprises a DNA.

Provided herein are compositions, wherein the composition comprises: a vector; and a nucleic acid, wherein the nucleic acid comprises a sequence comprising at least 85% sequence identity to SEQ ID NOS: 21, 22, 23, 24, 25, 26, or 271.

Provided herein are compositions, wherein the composition comprises: a vector; and a nucleic acid, wherein the nucleic acid encodes for a protein comprising a sequence comprising at least 85% sequence identity to SEQ ID NOS: 14, 15, 16, 17, 18, 19, or 20.

Provided herein are cells, wherein the cell comprises a composition as described herein. Further provided herein are cells, wherein the cell is an immune cell. Further provided herein are cells, wherein the immune cell is a lymphoid cell.

Provided herein are fusion proteins, wherein the fusion protein comprises: a TNF-superfamily ligand (TNFSF-L) or functional variant thereof; and a plurality of domains from a collectin family protein, wherein the plurality of domains from the collectin family protein comprises: an oligomerization domain or functional variant thereof; and a neck domain or functional variant thereof, wherein the oligomerization domain or functional variant thereof and the neck domain or functional variant thereof are closer in sequence proximity compared to their location in the collectin family protein. Further provided herein are fusion proteins, wherein the fusion protein comprises the following, in N-terminal to C-terminal order: the oligomerization domain or functional variant thereof; the neck domain or functional variant thereof; optionally, a linker sequence; and the TNFSF-L or functional variant thereof. Further provided herein are fusion proteins, wherein the TNFSF-L comprises Lymphotoxin alpha, OX40 ligand, CD40 ligand, Fas ligand, CD27 ligand, CD30 ligand, 4-1BBL, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. Further provided herein are fusion proteins, wherein the TNFSF-L is a CD40 ligand. Further provided herein are fusion proteins, wherein the CD40 ligand comprises at least 85% sequence identity to SEQ ID NO: 1. Further provided herein are fusion proteins, wherein the TNFSF-L is an OX40 ligand. Further provided herein are fusion proteins, wherein the OX40 ligand comprises at least 85% sequence identity to SEQ ID NO: 2 or SEQ ID NO: 3. Further provided herein are fusion proteins, wherein the TNFSF-L is a 4-1BBL. Further provided herein are fusion proteins, wherein the 4-1BBL comprises at least 85% sequence identity to SEQ ID NO: 4 or SEQ ID NO: 5. Further provided herein are fusion proteins, wherein the TNFSF-L is a LIGHT. Further provided herein are fusion proteins, wherein the LIGHT comprises at least 85% sequence identity to SEQ ID NO: 6. Further provided are compositions, wherein the TNFSF-L is a TNF superfamily member 18 ligand (GITRL). Further provided are compositions, wherein the GITRL comprises a sequence comprising at least 85% sequence identity to SEQ ID NO: 7 or SEQ ID NO: 8. Further provided herein are fusion proteins, wherein the collectin family protein comprises SP-A, SP-D, mannose binding lectin (MBL), conglutinin, CL-43, CL-L1, CL-K1, CL-P1, CL-46, or any combination thereof. Further provided herein are fusion proteins, wherein the collectin family protein is SP-D. Further provided herein are fusion proteins, wherein the oligomerization domain comprises at least 85% sequence identity to SEQ ID NO: 9. Further provided herein are fusion proteins, wherein the oligomerization domain and neck domain together comprise at least 85% sequence identity to SEQ ID NO: 10. Further provided herein are fusion proteins, wherein the oligomerization domain and neck domain together comprise SEQ ID NO: 10. Further provided herein are fusion proteins, wherein the fusion protein comprises the linker sequence, and wherein the linker sequence comprises GSG (glycine-serine-glycine) (SEQ ID NO: 12).

Provided herein are oncolytic viruses, wherein the oncolytic virus comprises: an exogenous nucleic acid encoding for a TNF-superfamily ligand (TNFSF-L) or functional variant thereof, wherein the TNFSF-L or functional variant thereof is fused to an oligomerization domain. Further provided herein are oncolytic viruses, wherein the oncolytic virus is Newcastle disease virus (NDV), Reovirus (RV), Myxoma virus (MYXV), Measles virus (MV), Herpes Simplex virus (HSV), Vaccinia virus (VV), Vesicular Somatitis virus (VSV), Polio virus (PV), Sendai virus, Flavivirus, Lentivirus, a pox virus, a retrovirus, an adeno-associated virus, or an adenovirus. Further provided herein are oncolytic viruses, wherein the nucleic acid encoding the TNFSF-L or functional variant thereof is inserted into the viral genome. Further provided herein are oncolytic viruses, wherein the nucleic acid encoding the TNFSF-L or functional variant thereof is inserted into a thymidine kinase gene. Further provided herein are oncolytic viruses, wherein the TNFSF-L comprises a Lymphotoxin alpha, OX40 ligand, CD40 ligand, Fas ligand, CD27 ligand, CD30 ligand, 4-1BBL, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. Further provided herein are oncolytic viruses, wherein the TNFSF-L is a CD40 ligand. Further provided herein are oncolytic viruses, wherein the CD40 ligand comprises a sequence comprising at least 85% sequence identity to SEQ ID NO: 1. Further provided herein are oncolytic viruses, wherein the TNFSF-L is an OX40 ligand. Further provided herein are oncolytic viruses, wherein the OX40 ligand comprises a sequence comprising at least 85% sequence identity to SEQ ID NO: 2 or SEQ ID NO: 3. Further provided herein are oncolytic viruses, wherein the TNFSF-L is a 4-1BBL. Further provided herein are oncolytic viruses, wherein the 4-1BBL comprises a sequence comprising at least 85% sequence identity to SEQ ID NO: 4 or SEQ ID NO: 5. Further provided herein are oncolytic viruses, wherein the TNFSF-L is a LIGHT. Further provided herein are oncolytic viruses, wherein the LIGHT comprises a sequence comprising at least 85% sequence identity to SEQ ID NO: 6. Further provided are compositions, wherein the TNFSF-L is a TNF superfamily member 18 ligand (GITRL). Further provided are compositions, wherein the GITRL comprises a sequence comprising at least 85% sequence identity to SEQ ID NO: 7 or SEQ ID NO: 8. Further provided herein are oncolytic viruses, wherein the oligomerization domain comprises a sequence comprising at least 85% sequence identity to SEQ ID NO: 9. Further provided herein are oncolytic viruses, wherein the nucleic acid comprises an RNA. Further provided herein are oncolytic viruses, wherein the nucleic acid comprises a DNA.

Provided herein are vaccinia viruses, wherein the vaccinia virus comprises: an exogenous nucleic acid encoding for a TNF-superfamily ligand (TNFSF-L) or functional variant thereof, wherein the TNFSF-L or functional variant thereof is fused to an oligomerization domain. Further provided herein are vaccinia viruses, wherein the vaccinia virus is a modified strain of Western Reserve Vaccinia virus (ATCC VR-1354), Copenhagen strain, Vaccinia virus Ankara (ATCC VR-1508), Vaccinia virus Ankara (ATCC VR-1566), recombinant vaccinia virus Ankara (MVA), NYVAC strain, Vaccinia virus strain Wyeth (ATCC VR-1536), Vaccinia virus Wyeth (ATCC VR-325), Wyeth (NYCBOH) strain, Tian Tan strain, Lister strain, USSR strain, and Evans strain. Further provided herein are vaccinia viruses, wherein the exogenous nucleic acid encoding the TNFSF-L or functional variant thereof is inserted into the viral genome. Further provided herein are vaccinia viruses, wherein the exogenous nucleic acid encoding the TNFSF-L or functional variant thereof is inserted into a thymidine kinase gene. Further provided herein are vaccinia viruses, wherein the TNFSF-L comprises a Lymphotoxin alpha, OX40 ligand, CD40 ligand, Fas ligand, CD27 ligand, CD30 ligand, 4-1BBL, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. Further provided herein are vaccinia viruses, wherein the TNFSF-L is a CD40 ligand. Further provided herein are vaccinia viruses, wherein the CD40 ligand comprises a sequence comprising at least 85% sequence identity to SEQ ID NO: 1. Further provided herein are vaccinia viruses, wherein the TNFSF-L is an OX40 ligand. Further provided herein are vaccinia viruses, wherein the OX40 ligand comprises a sequence comprising at least 85% sequence identity to SEQ ID NO: 2 or SEQ ID NO: 3. Further provided herein are vaccinia viruses, wherein the TNFSF-L is a 4-1BBL. Further provided herein are vaccinia viruses, wherein the 4-1BBL comprises a sequence comprising at least 85% sequence identity to SEQ ID NO: 4 or SEQ ID NO: 5. Further provided herein are vaccinia viruses, wherein the TNFSF-L is a LIGHT. Further provided herein are vaccinia viruses, wherein the LIGHT comprises a sequence comprising at least 85% sequence identity to SEQ ID NO: 6. Further provided are compositions, wherein the TNFSF-L is a TNF superfamily member 18 ligand (GITRL). Further provided are compositions, wherein the GITRL comprises a sequence comprising at least 85% sequence identity to SEQ ID NO: 7 or SEQ ID NO: 8. Further provided herein are vaccinia viruses, wherein the oligomerization domain comprises a sequence comprising at least 85% sequence identity to SEQ ID NO: 9. Further provided herein are vaccinia viruses, wherein the nucleic acid comprises an RNA. Further provided herein are vaccinia viruses, wherein the nucleic acid comprises a DNA.

Provided herein are methods for treatment of cancer, comprising: administering a pharmaceutical composition to a subject in an amount sufficient for treatment of a cancer, wherein the pharmaceutical composition comprises: a composition, cell, fusion protein, oncolytic virus, or vaccinia virus as described herein. Further provided herein are methods for treatment of cancer, wherein the cancer comprises a blood cancer or a solid cancer. Further provided herein are methods for treatment of cancer, wherein the cancer comprises a melanoma, a hepatocellular carcinoma, a breast cancer, a lung cancer, a peritoneal cancer, a prostate cancer, a bladder cancer, an ovarian cancer, a leukemia, a lymphoma, a renal carcinoma, a pancreatic cancer, an epithelial carcinoma, a gastric cancer, a colon carcinoma, a duodenal cancer, a pancreatic adenocarcinoma, a mesothelioma, a glioblastoma multiforme, an astrocytoma, a multiple myeloma, a prostate carcinoma, a hepatocellular carcinoma, a cholangiosarcoma, a pancreatic adenocarcinoma, a head and neck squamous cell carcinoma, a colorectal cancer, an intestinal-type gastric adenocarcinoma, a cervical squamous-cell carcinoma, an osteosarcoma, an epithelial ovarian carcinoma, an acute lymphoblastic lymphoma, a myeloproliferative neoplasm, or a sarcoma. Further provided herein are methods for treatment of cancer, wherein the cancer comprises a cancer of the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. Further provided herein are methods for treatment of cancer, wherein the administering comprises a systemic administration or a local administration. Further provided herein are methods for treatment of cancer, wherein the administering comprises an intratumoral administration, an intravenous administration, a regional administration, an intraperitoneal administration, a parenteral administration, an intramuscular administration, a subcutaneous administration, an intra-arterial administration, or any combination thereof. Further provided herein are methods for treatment of cancer, wherein the administering comprises an intratumoral administration. Further provided herein are methods for treatment of cancer, wherein the pharmaceutical composition further comprises a pharmaceutically acceptable carrier. Further provided herein are methods for treatment of cancer, wherein the pharmaceutically acceptable carrier comprises a buffer, an emulsion, a bioadsorbable polymer, a gel, or any combination thereof. Further provided herein are methods for treatment of cancer, wherein the composition, cell, or fusion protein is administered at a dose from about 0.01 μg/dose to about 1 g/dose. Further provided herein are methods for treatment of cancer, wherein the oncolytic virus or the vaccinia virus is administered at dose from about 10³ to about 10¹² PFU/dose. Further provided herein are methods for treatment of cancer, wherein the pharmaceutical composition is administered in a treatment cycle comprising 1, 2, 3, 4, 5, 6, 7, 8, 9, 10 or more doses. Further provided herein are methods for treatment of cancer, wherein the pharmaceutical composition is administered in each dose is administered over about 1 minute, about 1 hour, about 2 hours, about 3 hours, about 4 hours, about 1 day, or more. Further provided herein are methods for treatment of cancer, wherein each dose is independent of any other doses. Further provided herein are methods for treatment of cancer, wherein two or more doses in the treatment cycle are separated by a dose interval wherein no doses are administered. Further provided herein are methods for treatment of cancer, wherein the dose interval is about 1 minute, about 1 hour, about 2 hours, about 3 hours, about 4 hours, about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, or more. Further provided herein are methods for treatment of cancer, wherein each dose interval is independent of any other dose interval.

Provided herein are methods for reduction of tumor cell growth, comprising: administering to a tumor cell in an amount sufficient for reduction of tumor cell growth a composition, cell, fusion protein, oncolytic virus, or vaccinia virus as described herein. Further provided herein are methods for reduction of tumor cell growth, wherein the tumor comprises a liquid tumor or a solid tumor. Further provided herein are methods for reduction of tumor cell growth, wherein the tumor comprises a melanoma, a hepatocellular carcinoma, a breast tumor, a lung tumor, a peritoneal tumor, a prostate tumor, a bladder tumor, an ovarian tumor, a leukemia, a lymphoma, a renal carcinoma, a pancreatic tumor, an epithelial carcinoma, a gastric tumor, a colon carcinoma, a duodenal tumor, a pancreatic adenocarcinoma, a mesothelioma, a glioblastoma multiforme, an astrocytoma, a multiple myeloma, a prostate carcinoma, a hepatocellular carcinoma, a cholangiosarcoma, a pancreatic adenocarcinoma, a head and neck squamous cell carcinoma, a colorectal tumor, an intestinal-type gastric adenocarcinoma, a cervical squamous-cell carcinoma, an osteosarcoma, an epithelial ovarian carcinoma, an acute lymphoblastic lymphoma, a myeloproliferative neoplasm, or a sarcoma. Further provided herein are methods for reduction of tumor cell growth, wherein the tumor comprises a tumor of the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. Further provided herein are methods for reduction of tumor cell growth, wherein the administering comprises a systemic administration or a local administration. Further provided herein are methods for reduction of tumor cell growth, wherein the administering comprises intratumoral administration, intravenous administration, regional administration, intraperitoneal administration, parenteral administration, intramuscular administration, subcutaneous administration, intra-arterial administration, or any combination thereof. Further provided herein are methods for reduction of tumor cell growth, wherein the administering comprises an intratumoral administration. Further provided herein are methods for reduction of tumor cell growth, wherein the composition further comprises a pharmaceutically acceptable carrier. Further provided herein are methods for reduction of tumor cell growth, wherein the pharmaceutically acceptable carrier comprises a buffer, an emulsion, a bioadsorbable polymer, a gel, or any combination thereof. Further provided herein are methods for reduction of tumor cell growth, wherein the composition, cell, or fusion protein is administered at a dose from about 0.01 μg/dose to about 1 g/dose. Further provided herein are methods for reduction of tumor cell growth, wherein the oncolytic virus or the vaccinia virus is administered at dose from about 10³ to about 10¹² PFU/dose. Further provided herein are methods for reduction of tumor cell growth, wherein the administering is in a treatment cycle comprising 1, 2, 3, 4, 5, 6, 7, 8, 9, 10 or more doses. Further provided herein are methods for reduction of tumor cell growth, wherein each dose is administered over about 1 minute, about 1 hour, about 2 hours, about 3 hours, about 4 hours, about 1 day, or more. Further provided herein are methods for reduction of tumor cell growth, wherein two or more doses are separated by a dose interval wherein no doses are administered. Further provided herein are methods for reduction of tumor cell growth, wherein the dose interval is about 1 minute, about 1 hour, about 2 hours, about 3 hours, about 4 hours, about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, or more. Further provided herein are methods for reduction of tumor cell growth, wherein each dose interval is independent of any other dose interval

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative embodiments, in which the principles of this disclosure are utilized, and the accompanying drawings of which:

FIG. 1 illustrates a diagram of an exemplary domain structure having an oligomerization domain, neck domain, optional linker, and TNFSF-L domain.

FIG. 2 shows expression of a CD40L scaffold protein in cells infected with a modified oncolytic vaccinia virus transgene expressing the CD40L scaffold protein compared to cells infected with an oncolytic vaccinia virus not expressing the transgene or uninfected cells. The Y-axis accounts for mCD40L in ng/mL and the X-axis accounts for sample (uninfected cells, vaccinia virus having A52R deletion and thymidine kinase deletion, and vaccinia virus having thymidine kinase deletion and insertion of a variant CD40L).

FIG. 3 shows results from an mOX40L ELISA assay, where supernatant is diluted 100× after accounting for dilution factor and screened for mOX40L expression. The Y-axis accounts for mOX40L in μg/mL and the X-axis accounts for sample (uninfected cells, vaccinia virus having A52R deletion and thymidine kinase deletion, vaccinia virus having thymidine kinase deletion and native OX40L, and vaccinia virus having thymidine kinas deletion and insertion of a variant OX40L).

FIG. 4 shows results from a 41BBL ELISA assay, where undiluted supernatant is screened for 41BBL expression. The Y-axis accounts for m41BBL in ng/mL and the X-axis accounts for sample (uninfected cells, vaccinia virus having A52R deletion and thymidine kinase deletion, vaccinia virus having thymidine kinase deletion and native 41BBL, and vaccinia virus having thymidine kinas deletion and insertion of a variant 41BBL).

FIG. 5 shows CD40L reporter cell assay results. The Y-axis is OD and the X-axis is the volume of supernatant used. Samples are uninfected cells, cells infected with a vaccinia virus having an A52R deletion and thymidine kinase deletion, and cells infected with a vaccinia virus comprising a thymidine kinase deletion and a variant CD40L.

FIG. 6 shows probability of survival in mice treated with virus having a TK deletion (TK-), virus having a TNFSFL fusion construct (s3CD40L), or a buffer control.

FIG. 7 shows luciferase expression levels (RLU) in uninfected control cells (buffer), cells infected with virus expressing 41BBL, and cells infected with virus expressing trimeric OX40L (3sOX40L).

FIG. 8 shows luciferase expression levels (RLU) in uninfected control cells (buffer), cells infected with: virus expressing trimeric GITRL as a negative control, virus expressing 41BBL monomer, and virus expressing trimeric 41BBL (3s41BBL).

FIG. 9 shows IL-2 expression levels in CD8 T-cells stimulated with CD3 alone, or CD3 with: CD28, recombinant 41BBL, recombinant OX40L, virus expressing 3s41BBL, or virus expressing 3xOX40L.

FIG. 10 shows average tumor volume in BALB/c mice with subcutaneous RENCA tumors with volume in mm³ on the y-axis measured in days post treatment on the x-axis. Volume was measured at 0, 5, 8, and 12 days post treatment with buffer control, control vaccinia virus with a Thymidine Kinase deletion (HCCTKM), virus expressing 3s41BBL, or virus expressing 3sOX40L. Tumor volume in treatment groups comprising virus expressing ligands 3s41BBL or 3sOX40L showed smaller tumor volumes compared to negative controls after 5, 8. And 12 days.

FIG. 11A shows a scatter plot of tumor volume in mm³ in BALB/c mice with subcutaneous Lewis Lung Carcinoma (LLC) tumors 30 days after treatment with buffer control, control vaccinia virus with a Thymidine Kinase deletion (HCCTKM), or virus expressing 3sGITRL. Average tumor volumes for each group after 30 days are indicated, showing about 1400 mm³ in the buffer control group, about 750 mm³ for the HCCTKM group, and <100 mm³ for the 3sGITRL group.

FIG. 11B shows a scatter plot of tumor volume in mm³ in BALB/c mice with subcutaneous RENCA tumors 17 days after treatment with buffer control, control vaccinia virus with a Thymidine Kinase deletion (HCCTKM), or virus expressing 3sGITRL. Average tumor volumes for each group after 17 days are indicated, showing about 800 mm³ in the buffer control group, about 350 mm³ in the HCCTKM group, and about 250 mm³ in the 3sGITRL group.

### DETAILED DESCRIPTION

While preferred embodiments of this disclosure have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments of this disclosure described herein may be employed in practicing the disclosure. It is intended that the following claims define the scope of the disclosure and that methods and structures within the scope of these claims and their equivalents be covered thereby.

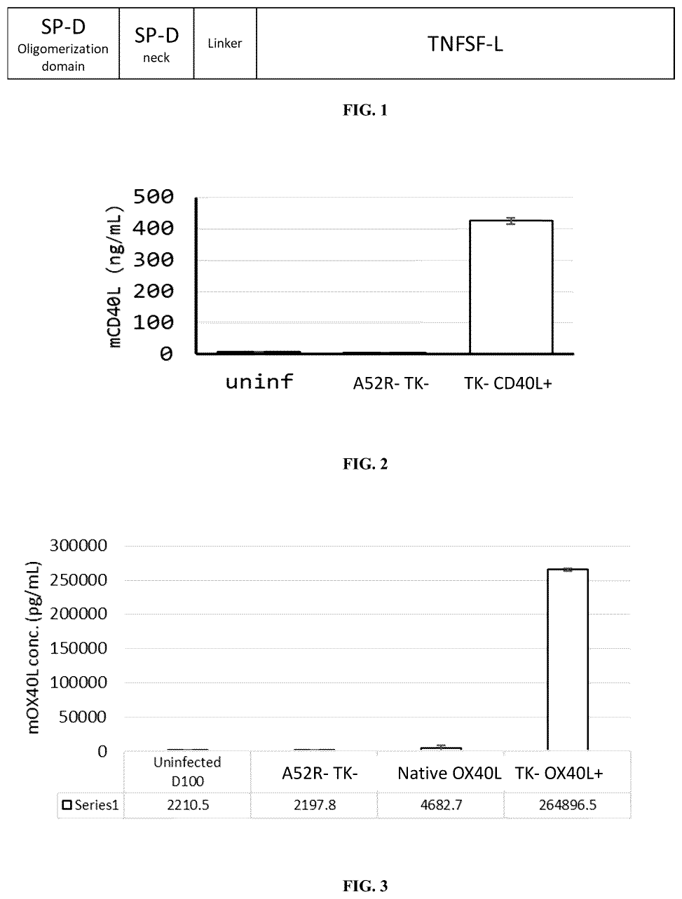

Provided herein are compositions, and uses thereof for treatment of cancer, comprising a nucleic acid coding for a fusion protein of a TNF superfamily ligand (TNFSF-L) or functional variant thereof fused to a polymerization domain. In one exemplary arrangement, with reference to FIG. 1, a TNFSF-L is fused (optionally via a linker) to an N-terminal oligomerization domain and, optionally, a neck domain C-terminal to the oligomerization domain. In the illustration of FIG. 1, the oligomerization domain and neck domain are functional domains from a collectin family protein, SP-D. The additional oligomerization domain, and optional neck domain and/or linker domain, allow for polymerization of the fusion protein providing for structures having increased activity in triggering an immune response, or markers thereof. Described in more detail herein are TNFSF-Ls, domains for fusion to the TNFSF-L, viral and non-viral vectors for delivery of nucleic acid encoding the fusion proteins, conditions for use of the compositions, and methods of administration. Further provided herein are composition and methods for treatment of cancer generally including an oncolytic virus comprising a nucleic acid encoding a TNFSF-L fusion protein. Additionally provided herein are cells comprising a nucleic acid expressing a fusion protein as described herein.

### Definitions

The terminology used herein is for the purpose of describing particular cases only and is not intended to be limiting. As used herein, the singular forms “a”, “an” and “the” can include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, e.g., the limitations of the measurement system. Where particular values are described in the application and claims, unless otherwise stated the term “about” should be assumed to mean an acceptable error range for the particular value, such as ±10% of the value modified by the term “about”.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The term “mutation,” as used herein, can refer to a deletion, an insertion of a heterologous nucleic acid, an inversion or a substitution, including an open reading frame ablating mutations as commonly understood in the art.

The term “gene,” as used herein, can refer to a segment of nucleic acid that encodes an individual protein or RNA (also referred to as a “coding sequence” or “coding region”), optionally together with associated regulatory regions such as promoters, operators, terminators and the like, which may be located upstream or downstream of the coding sequence.

A “promoter,” as used herein, can be a control sequence that is a region of a nucleic acid sequence at which initiation and rate of transcription are controlled. In some embodiments, a promoter may comprise genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In some embodiments, a promoter may or may not be used in conjunction with an “enhancer,” which refers to a cis-acting regulatory sequence involved in the transcriptional activation of a nucleic acid sequence.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA.

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself.

The term “effective amount” or “therapeutically effective amount” can refer to the amount of a compound that, when administered, can be sufficient to prevent development of, or alleviate to some extent, one or more of the symptoms of the disorder, disease, or condition being treated.

The term “oncolytic,” as used herein, can refer to killing of cancer or tumor cells by an agent, such as an oncolytic poxvirus, such as an oncolytic vaccinia virus, e.g., through the direct lysis of said cells, by stimulating immune response towards said cells, apoptosis, expression of toxic proteins, autophagy and shut-down of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” can refer to killing of cancer or tumor cells without lysis of said cells.

The term “oncolytic virus” as used herein can refer to a virus that preferentially infects and kills tumor cells.

The term “modified oncolytic virus” as used herein can refer to an oncolytic virus that comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of a exogenous protein or modified viral protein to the viral capsid. In general, oncolytic viruses may be modified (also known as “engineered”) in order to gain improved therapeutic effects against tumor cells.

### Fusion Constructs

Provided herein are nucleic constructs encoding for fusion proteins. In some embodiments, a fusion protein described herein comprises a TNFSF-L region and an oligomerization region, optionally from a collectin protein. Such fusion proteins may be coded by nucleic acids and inserted into an oncolytic virus. The resultant expressed fusion protein may further comprise a linker region between the TNFSF-L region and the oligomerization region. Examples of TNF superfamily members for inclusion in the TNFSF-L region include, without limitation: Lymphotoxin alpha, OX40 ligand, CD40 ligand, Fas ligand, CD27 ligand, CD30 ligand, CD137 ligand, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. In further embodiments, the TNFSF-L region can be linked at the N terminus to a linker peptide. In some instances, linker peptides comprise up to 10 amino acids in length and, optionally, are glycine and/or serine rich.

In some embodiments, provided herein are the fusion proteins themselves. In some embodiments, the fusion protein comprises a TNFSF-L region and an oligomerization region, optionally from a collectin protein. Such fusion proteins may be coded by nucleic acids and inserted into an oncolytic virus. The resultant expressed fusion protein may further comprise a linker region between the TNFSF-L region and the oligomerization region. Examples of TNF superfamily members for inclusion in the TNFSF-L region include, without limitation: Lymphotoxin alpha, OX40 ligand, CD40 ligand, Fas ligand, CD27 ligand, CD30 ligand, CD137 ligand, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. In further embodiments, the TNFSF-L region can be linked at the N terminus to a linker peptide. In some instances, linker peptides comprise up to 10 amino acids in length and, optionally, are glycine and/or serine rich.

Amino acid sequences of exemplary Tumor Necrosis Superfamily Ligands for inclusion in whole or in part in fusion constructs described herein are indicated in Table 1. Such exemplary TNFSF-L proteins include ectodomains from murine CD40L, human OX40L, murine OX40L, murine 4-1BBL, human 4-1BBL, human GITRL, murine GITRL, murine 3sCD40L, murine 3sOX40L, murine 3s4-1BBL, murine 3sGITRL, human 3sOX40L, human 3s4-1BBL, and murine 3sGITRL.

TABLE 1

| SEQ ID | | |
|---|---|---|
| NO: | Description | Amino Acid Sequence |
| 1 | CD40-L | HRRLDKVEEEVNLHEDFVFIKKLKRCNKGEGSLSLLNCEEMRRQF |
| | (murine) | EDLVKDITLNKEEKKENSFEMQRGDEDPQIAAHVVSEANSNAASV |
| | | LQWAKKGYYTMKSNLVMLENGKQLTVKREGLYYVYTQVTFCSNRE |
| | | PSSQRPFIVGLWLKPSSGSERILLKAANTHSSSQLCEQQSVHLGG |
| | | VFELQAGASVFVNVTEASQVIHRVGFSSFGLLKL |
| 2 | OX40-L | RIQSIKVQFTEYKKEKGFILTSQKEDEIMKVQNNSVIINCDGFYL |
| | (human) | ISLKGYFSQEVNISLHYQKDEEPLFQLKKVRSVNSLMVASLTYKD |
| | | KVYLNVTTDNTSLDDFHVNGGELILIHQNPGEFCVL |
| 3 | OX40-L | AKDPPIQRLRGAVTRCEDGQLFISSYKNEYQTMEVQNNSVVIKCD |
| | (murine) | GLYIIYLKGSFFQEVKIDLHFREDHNPISIPMLNDGRRIVFTVVA |
| | | SLAFKDKVYLTVNAPDTLCEHLQINDGELIVVQLTPGYCAPEGSY |
| | | HSTVNQVPL |
| 4 | 4-1BBL | SHIGCPNTTQQGSPVFAKLLAKNQASLCNTTLNWHSQDGAGSSYL |
| | (murine) | SQGLRYEEDKKELVVDSPGLYYVFLELKLSPTFTNTGHKVQGWVS |
| | | LVLQAKPQVDDFDNLALTVELFPCSMENKLVDRSWSQLLLLKAGH |
| | | RLSVGLRAYLHGAQDAYRDWELSYPNTTSFGLFLVKPDNPWE |
| 5 | 4-1BBL | QGMFAQLVAQNVLLIDGPLSWYSDPGLAGVSLTGGLSYKEDTKEL |
| | (human) | VVAKAGVYYVFFQLELRRVVAGEGSGSVSLALHLQPLRSAAGAAA |
| | | LALTVDLPPASSEARNSAFGFQGRLLHLSAGQRLGVHLHTEARAR |
| | | HAWQLTQGATVLGLFRVTPEIPAGLPSPRSE |
| 6 | LIGHT | MEESVVRPSVFVVDGQTDIPFTRLGRSHRRQSCSVARVGLGLLLL |
| | (human) | LMGAGLAVQGWFLLQLHWRLGEMVTRLPDGPAGSWEQLIQERRSH |
| | | EVNPAAHLTGANSSLTGSGGPLLWETQLGLAFLRGLSYHDGALVV |
| | | TKAGYYYTYSKVQLGGVGCPLGLASTITHGLYKRTPRYPEELELL |
| | | VSQQSPCGRATSSSRVWWDSSFLGGVVHLEAGEKVVVRVLDERLV |
| | | RLRDGTRSYFGAFMV |
| 7 | GITRL | TSLKPTAIESCMVKFELSSSKWHMTSPKPHCVNTTSDGKLKILQS |
| | (mouse) | GTYLIYGQVIPVDKKYIKDNAPFVVQIYKKNDVLQTLMNDFQILP |
| | | IGGVYELHAGDNIYLKFNSKDHIQKTNTYWGIILMPDLPFIS |
| 8 | GITRL | TSLKPTAIESCMVKFELSSSKWHMTSPKPHCVNTTSDGKLKILQS |
| | (human) | GTYLIYGQVIPVDKKYIKDNAPFVVQIYKKNDVLQTLMNDFQILP |
| | | IGGVYELHAGDNIYLKFNSKDHIQKTNTYWGIILMPDLPFIS |

Many TNF superfamily receptors blind their cognate ligands as homotrimers. Ligands are natively expressed as transmembrane anchored proteins on the surface of cells. Provided herein are fusion constructs comprising TNFSF ligands fused to an oligomerization-promoting fragment, such as from a human collectin protein. In some embodiments, the oligomerization region comprises a collectin protein polypeptide fragment, optionally linked to the N terminus of the TNFSF-L region, or to the N terminus of the linker peptide, wherein the C terminus of the linker peptide is linked to the N terminus of the TNFSF-L region. Typically, collectins comprise a C-terminal carbohydrate section, a neck section, a collagenous section, and an N-terminal section. The C-terminal carbohydrate region of collectins, also known as the lectin domain or CRD typically comprising cystine residues to aid on oligomerization. The neck section initiates trimerization and forms a zipper like fashion along the collagenous tail of the neck section towards the N-terminal section. The N-terminal section itself includes a cystine rich portion that aids in oligomerization through disulfide bridges.

### Oligomerization Domains

Provided herein are TNFSF-L fusion constructs comprising an oligomerization region. Such regions allow for the enhanced stability and functionality of expressed TNFSF-L, and therefore for increased activation of host immune system activity. In some embodiments, the oligomerization region comprises at least one domain or functional variant thereof from a collectin family protein. Collectin proteins are known to form stable oligomers. Collectins are one of 18 group members building the protein lectin superfamily comprising a structural protein fold called C-type lectin domain. Some members have been shown to comprise additional structural features; thus, they comprise the following components: i) an N-terminal collagen domain connected to ii) an alpha-helical segment that is also referred to as the neck-region and iii) the CRD at the C-terminus. Examples of collectins include surfactant protein A (SP-A), surfactant protein D (SP-D), mannose binding lectin (MBL), conglutinin, CL-43, CL-L1, CL-K1, CL-P1, and CL-46. SP-A and SP-D comprise N-terminal cysteines that are involved in the disulfide-mediated oligomerization of pre-formed trimers. In some embodiments, nucleic acids encoding peptide sequences above are also contemplated. In some embodiments, a fusion construct described herein comprises a collectin oligomerization domain or functional variant thereof. In some embodiments, a fusion construct described herein comprises a collectin oligomerization domain or functional variant thereof and a collectin neck domain or functional variant thereof. Exemplary sequences are provided in Table 2 (amino acids) and Table 3 (nucleic acids).

TABLE 2

| SEQ ID | | |
|---|---|---|
| NO: | Description | Amino Acid Sequence |
| 9 | oligomerization | MLLFLLSALVLLTQPLGYL |
| | domain from SP-D | EAEMKTYSHRIMPSACTLV |
| | | MCSSVESGLPGRDG |
| 10 | oligomerization | MLLFLLSALVLLTQPLGYL |
| | domain and neck | EAEMKTYSHRTMPSACTLV |
| | from SP-D (bold | MCSSVESGLPGRDGSDVAS |
| | for oligomeri- | LRQQVEALQGQVQHLQAAF |
| | zation, non-bold | SQYKKVE |
| | for neck) | |

TABLE 3

| SEQ ID | | Nucleic Acid |
|---|---|---|
| NO: | Description | Sequence |
| 11 | oligomerization | ATGCTGCTCTTCCTCC |
| | domain and neck | TCTCTGCACTGGTCCT |
| | domain from | GCTCACACAGCCCCTG |
| | SP-D | GGCTACCTGGAAGCAG |
| | | AAATGAAGACCTACTC |
| | | CCACAGAACAATGCCC |
| | | AGTGCTTGCACCCTGG |
| | | TCATGTGTAGCTCAGT |
| | | GGAGAGTGGCCTGCCT |
| | | GGTCGCGATggatctg |
| | | atGTTGCTTCTCTGAG |
| | | GCAGCAGGTTGAGGCC |
| | | TTACAGGGACAAGTAC |
| | | AGCACCTCCAGGCTGC |
| | | TTTCTCTCAGTATAAG |
| | | AAAgtGgaG |

In some embodiments, a collectin polypeptide fragment is linked to the N terminus of the of a TNFSF-L region of the fusion protein, or to the N terminus of the linker peptide, wherein the C terminus of the linker peptide is linked to the N terminus of the TNFSF-L region the fusion protein.

### Linkers

Provided herein are fusion constructs comprising a flexible linker element located between an oligomerization domain and a TNFSF-L domain. In some embodiments, the flexible linker element has a length of 25 amino acids or less. In some embodiments the linker element has a length of 3-30 amino acids, particularly a length of 3, 4, 5, 6, 7, 8, 9, 10, 11, 12, 13, 14, 15, 16, 17, 18, 19, 20, 21, 22, 23, 24, 25, 26, 27, 28 or 30 amino acids. In some embodiments, the length of the linker comprises 3-10 or 5-10 amino acids. In some embodiments the linker elements are built of small and hydrophilic non-charged amino acids. In some embodiments, the linker element according to the invention may comprise amino acids selected from G, S, A and T. The linker element is preferably a glycine/serine linker, i.e., a peptide linker comprising the amino acids glycine and serine. In some embodiments, the linker comprises the amino acid sequence (GSG) (SEQ ID NO: 12). In some embodiments, the linker comprises the amino acid sequence (GSS)a(SSG)b(GSG)c (SEQ ID NO: 13) wherein a, b, c is each 0, 1, 2, 3, 4, 5 or 6 repeats. If a TNFSF-L ends or begins with a serine or glycine amino acid, such a residue may also form the first residue of the linker. Building blocks for the liker elements may be composed of 1, 2, 3, 4, 5 or more amino acids. Generally, a linker element as used herein may be composed of building blocks or also from a sequence of amino acids.

### Delivery Vectors

Provided herein are systems to delivery of nucleic acids encoding fusion constructs as described herein provides for entry the nucleic acid construct into a target cell while reducing nuclease degradation of the nucleic acid. Such delivery systems may comprise a viral or a non-viral vector.

### Oncolytic Viruses

Provided herein are vectors in the form of virus. In some embodiments, viral delivery vectors comprise oncolytic viruses. Provided herein are compositions comprising an oncolytic virus wherein the oncolytic virus comprises a TNFSF-L fusion construct described herein. Oncolytic viruses as used herein, kill cancer or tumor cells through mechanisms such as the direct lysis of said cells, by stimulating immune response towards said cells, apoptosis, expression of toxic proteins, autophagy and shut-down of protein synthesis, induction of anti-tumoral immunity, or any combinations thereof. Exemplary oncolytic viruses for inclusion in composition described herein include, without limitation, a pox virus, Newcastle disease virus (NDV), Reovirus (RV), Myxoma virus (MYXV), Measles virus (MV), Herpes Simplex virus (HSV), Vaccinia virus (VV), Vesicular Stomatitis virus (VSV), Polio virus (PV), Sendai virus, Flavivirus, Lentivirus, retroviruses, adeno-associated viruses, and adenoviruses. In some embodiments, the oncolytic virus can be a poxvirus. In some embodiments, the poxvirus comprises a betaentomopoxvirus, a yatapoxvirus, a cervidpoxvirus, a gammaentomopoxvirus, a leporipoxvirus, a suipoxvirus, a molluscipoxvirus, a crocodylidpoxvirus, an alphaentomopoxvirus, a capripoxvirus, an avipoxvirus, parapoxvirus, a canarypoxvirus, or a fowlpox virus. These oncolytic viruses have a proclivity to specifically target cancer cells, and upon virus replication cause significant cell death and tumor regression.

In some embodiments, the oncolytic virus can be a modified oncolytic virus that can have one or more modifications that can result in a greater therapeutic effect against tumor cells, as compared to an otherwise identical virus that does not comprises the modifications. Some non-limiting examples of the greater therapeutic effect may include each or any combinations of: enhanced immune evasion of the virus, enhanced tumor-targeted systemic delivery of the virus, enhanced intratumoral and intertumoral spreading of the virus, and enhanced tumor-specific replication of the virus, or release of immune modulators and anti-tumor agents into the extracellular matrix. The modified oncolytic virus of this disclosure, in some instances, can be utilized as a platform vector for systemic delivery.

In some embodiments of the present disclosure, provided is a modified oncolytic virus comprising a modification that can enhance tumor-targeted systemic delivery of the virus. Typically, oncolytic viruses can be either be (a) administered systemically or (b) inoculated topically over the tumor or, in many cases, injected directly into the tumor (“intratumoral delivery”). It is believed that systemic delivery of the oncolytic virus can afford the opportunity to treat both the primary tumor and any overt or undiagnosed metastatic deposits simultaneously. As a result, this method of delivery can be a very attractive option for the treatment of patients with advanced/metastatic disease or patients with inaccessible disease such as those with pancreatic cancer or brain cancer, where access is difficult for example due to physiological barriers, such as blood-brain barrier. However, barriers can exist for successful systemic delivery of many oncolytic viruses. For instance, in some cases, as described above, host defense limits most oncolytic viruses' ability to infect tumors after systemic administration. Blood cells, complement, antibodies, and antiviral cytokines, as well as nonspecific uptake by other tissues such as the lung, liver and spleen, tissue-resident macrophages, and additionally poor virus escape from the vascular compartment are among the main barriers to systemic delivery of oncolytic viruses. In some embodiments of the present disclosure, disclosed oncolytic viruses can comprise a modification that can promote the persistent existence of the virus in the circulation system, at least through, as abovementioned, enhancement of immune evasion. On the other hand, enhanced tumor-targeted delivery of the virus can also be desirable under certain circumstances, as it may not only increase therapeutic efficacy against cancer, but may also alleviate the safety concerns around virus-mediated oncotherapy as the non-tumor infection can be limited, avoiding the undesired side effects of viral infection. Some embodiments herein relate to an oncolytic virus comprising a modification that can promote the tumor-targeted delivery of the virus.

In some embodiments of the present disclosure, provided is a modified oncolytic virus comprising a modification that can enhance intratumoral and intertumoral spreading of the virus. Enhanced spreading of the oncolytic virus within and between tumors can boost the therapeutic efficacy by increasing the number of the cancer cells that are infected by the virus. Provided herein, in some embodiments, is a modified oncolytic virus that can comprise an exogenous nucleic acid. Provided herein, in some embodiments, is a modified oncolytic virus that can comprise a modification to in the genome of the virus. Provided herein, in some embodiments, is a modified oncolytic virus that can comprise an exogenous nucleic acid as well as a modification in the genome of the virus.

In some embodiments, the oncolytic viruses can include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some embodiments, the oncolytic virus can be a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a retrovirus, a mengovirus, or a myxoma virus. In some embodiments, the oncolytic virus can be a pox virus. In some embodiments, the pox virus can be a vaccinia virus. In some cases the modified pox virus can be an attenuated canarypox virus. In some cases the modified pox virus can be a fowlpox virus.

In some embodiments, a modified oncolytic virus is employed. In general such a virus comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of a exogenous protein or modified viral protein to the viral capsid.

In some embodiments, the modified oncolytic virus can comprise an exogenous nucleic acid that can code for LIGHT. In some embodiments, the modified oncolytic virus can comprise an exogenous nucleic acid that can code for IL15. In some embodiments, the modified oncolytic virus can comprise an exogenous nucleic acid that can code for IL15, and exogenous nucleic acid that can code for CCL5. In some embodiments, the modified oncolytic virus can comprise an exogenous nucleic acid that can code for IL15, and exogenous nucleic acid that can code for IL15-Rα. In some embodiments, the modified oncolytic virus can comprise an exogenous nucleic acid that can code for ITAC (CXCL11), and an exogenous nucleic acid that can code for a fractalkine (CX3CL1). In some embodiments, the modified oncolytic virus can comprise an exogenous nucleic acid that can code for ITAC (CXCL11), an exogenous nucleic acid that can code for a fractalkine (CX3CL1), an exogenous nucleic acid that can code for IL15, and exogenous nucleic acid that can code for IL15-Rα.

In some embodiments, the modified oncolytic virus can comprise a mutation or deletion of the K7R gene and can further comprise an exogenous nucleic acid that can code for a cytokine, e.g., IL15. In some embodiments, the modified oncolytic virus can comprise a mutation or deletion of the K7R gene, and can further comprise an exogenous nucleic acid that can code for a cytokine, e.g., IL15, and an exogenous nucleic acid that can code for a chemokine, e.g., CCL5. In some embodiments, the modified oncolytic virus can comprise a mutation or deletion of the K7R gene, and can further comprise an exogenous nucleic acid that can code for a cytokine, e.g., IL15, and an exogenous nucleic acid that can code for a receptor for the cytokine, e.g., IL15Rα. In some embodiments, the modified oncolytic virus can comprise a mutation or deletion of the K7R gene and can further comprise exogenous nucleic acid that can code for LIGHT. In some embodiments, the modified oncolytic virus can comprise a mutation or deletion of the K7R gene and can further comprise an exogenous nucleic acid that can code for ITAC (CXCL11). In some embodiments, the modified oncolytic virus can comprise a mutation or deletion of the K7R gene and comprise an exogenous nucleic acid that can code for a fractalkine (CX3CL1). In some embodiments, the modified oncolytic virus can comprise a mutation or deletion of the K7R gene and can further comprise an exogenous nucleic acid that can code for ITAC (CXCL11), and an exogenous nucleic acid that can code for a fractalkine (CX3CL1).

In some embodiments, the modified oncolytic virus can comprise a mutation or deletion of the A52R gene and can further comprise an exogenous nucleic acid that can code for a cytokine, e.g., IL15. In some embodiments, the modified oncolytic virus can comprise a mutation or deletion of the A52R gene, and can further comprise an exogenous nucleic acid that can code for a chemokine, e.g., IL15, and an exogenous nucleic acid that can code for a chemokine, e.g., CCL5. In some embodiments, the modified oncolytic virus can comprise a mutation or deletion of the A52R gene, and can further comprise an exogenous nucleic acid that can code for a cytokine, e.g., IL15, and an exogenous nucleic acid that can code for a receptor for the cytokine, e.g., IL15Rα. In some embodiments, the modified oncolytic virus can comprise a mutation or deletion of the A52R gene and can further comprise exogenous nucleic acid that can code for LIGHT. In some embodiments, the modified oncolytic virus can comprise a mutation or deletion of the A52R gene and can further comprise an exogenous nucleic acid that can code for ITAC (CXCL11). In some embodiments, the modified oncolytic virus can comprise a mutation or deletion of the A52R gene and comprise an exogenous nucleic acid that can code for a fractalkine (CX3CL1). In some embodiments, the modified oncolytic virus can comprise a mutation or deletion of the A52R gene and can further comprise an exogenous nucleic acid that can code for ITAC (CXCL11), and an exogenous nucleic acid that can code for a fractalkine (CX3CL1).

In some examples, the co-expression of a cytokine (e.g., IL15) and its receptor (e.g., IL15-Rα), from the modified oncolytic virus, can, in some cases, lead to enhanced immunomodulatory effects of the oncolytic virus, for example, due to improved ability of a complex formed by IL15 and IL15-Rα (IL15:IL15-R complex) to activate natural killer cells and promote T cell response. Without being bound by any specific theory, it is contemplated that the IL15 in the IL15:IL15-Rα complex can be presented to the IL15-Rβγ (IL15-receptor beta gamma complex) displayed on the surface of T cells and natural killer (NK) cells, thereby imparting potent immunomodulatory effects on the NK cells and the T cells.

In some embodiments are provided modified oncolytic viruses wherein the A52R gene can be mutated or deleted, and further, wherein the modified oncolytic viruses can comprise an exogenous nucleic acid that can code for a secreted hyaluronidase, such as HysA. In some embodiments are provided modified oncolytic viruses wherein the A52R gene can be mutated or deleted, and further, wherein the modified oncolytic viruses can comprise an exogenous nucleic acid that can code for a chemokine receptor, such as CXCR4.

In some embodiments, the modified oncolytic virus can comprise a complete or a partial deletion of the viral thymidine kinase (TK) gene. In some embodiments, in the genome of the modified oncolytic virus disclosed herein, one or more of the exogenous nucleic acids are inserted in the loci of the deleted TK gene.

In some embodiments, in the modified oncolytic virus, such as in an oncolytic vaccinia virus, the viral TK gene may be replaced with a TK gene from a herpes simplex virus (HSV-TK). The HSV TK may function as a substitute for the deleted TK and may have multifaceted advantages. For instance, (i) HSV TK can be used as an additional therapeutic prodrug converting enzyme for converting ganciclovir (GCV) into its cytotoxic metabolite in a tumor. In addition to the added therapeutic effect this modification can also serve as a suicide gene, e.g., vaccinia expressing cells can be killed efficiently through addition of GCV, thereby shutting down the virus in the case of an adverse event or uncontrolled replication. Thus, in some instances, the modified oncolytic virus of this disclosure can act as a safety switch). In additional examples, a mutated version of the HSV TK can be used to allow for PET imaging of labelled substrates with greatly increased sensitivity. Thus, in some cases, the modified oncolytic virus, comprising an HSV TK that can be used in PET imaging, can act as a reporter of viral replication in vivo to determine therapeutic activity early after treatment.

In some cases, the modified oncolytic virus can comprise a full-length viral backbone gene or viral backbone protein described above, or truncated versions thereof, or functional domains thereof, or fragments thereof, or variants thereof. In various examples, the modified oncolytic virus can comprise mutation or deletion of one or more of viral backbones genes or viral backbone proteins, as described above. Mutations of the viral backbone genes and viral backbone proteins can comprise insertion, deletion, substitution, or modifications of nucleotides in nucleic acid sequences and amino acids in protein sequences. Deletion can comprise, in some examples, a complete or partial deletion of the viral backbone gene or protein.

### Vaccinia Viruses

In some embodiments, the oncolytic virus is a vaccinia virus. Exemplary vaccinia viruses include, without limitation, a wild type or attenuated vaccinia virus strain modified by inclusion of a fusion construct described herein, such as Western Reserve Vaccinia virus (ATCC VR-1354), Copenhagen strain, Vaccinia virus Ankara (ATCC VR-1508), Vaccinia virus Ankara (ATCC VR-1566), recombinant vaccinia virus Ankara (MVA), NYVAC strain, Vaccinia virus strain Wyeth (ATCC VR-1536), Vaccinia virus Wyeth (ATCC VR-325), Wyeth (NYCBOH) strain, Tian Tan strain, Lister strain, USSR strain, and Evans strain. The base vaccinia virus strain modified as set forth herein may itself comprise one or more mutation relative to its parent strain, for example, but not limited to, one or more of the following: deletion in TK (also referred to herein as “TK-”) and deletion in A52R (also referred to herein as “A52R-”). Vaccinia viruses may be recombinant or selected to have low toxicity and to accumulate in the target tissue. In some embodiments, the modifications in the viral backbone/viral genome are modifications that render the vaccinia virus non-replicating or comprise a poor replicative capacity. Non-limiting examples of such modifications can include mutations in the following viral genes: A1, A2, VH1, A33, and I7. In some embodiments, the viral backbone mutation is selected from the group consisting of: a complete or partial deletion of the A52R gene; a complete or partial deletion of the TK gene; a complete or partial deletion of the B15R gene; a complete or partial deletion of the K7R gene; a complete or partial deletion of the B14R gene; a complete or partial deletion of the N1L gene; a complete or partial deletion of the K1L gene; a complete or partial deletion of the M2L gene; a complete or partial deletion of the A49R gene; a complete or partial deletion of the VH1 gene; a complete or partial deletion of A33 gene; a complete or partial deletion of A1; a complete or partial deletion of A2 gene; a complete or partial deletion of 17 gene, and a complete or partial deletion of the A46R gene. As used herein, the reference to a viral gene can be made by reference to the protein encoded by the gene (e.g., A33 gene can mean a gene that codes for the A33 protein). In some embodiments, the viral backbone mutation, including any combinations of substitution, insertion, and deletion, can result in a sequence with less than 100%, 99%, 98,%, 97%, 96%, 95%, 94%, 93%, 92%, 91%, 90% or less sequence homology to the wild-type sequence of the viral gene or a viral protein coded by the gene. The viral gene and protein coded by the same, in some embodiments, is selected from the group consisting of: B15R, K7R, B14R, N1L, K1L, M2L, A49R, VH1, A33, A1, A2, 17, and A46R. In some embodiments, the viral backbone can comprise 1, 2, 3, 4, 5, or more mutations in the amino acid sequence of the viral protein (e.g., a viral antigen). The viral antigen is in some examples selected from the group consisting of: B15R, K7R, B14R, N1L, K1L, M2L, A49R, VH1, A33, A1, A2, 17, and A46R The disclosure provides in some embodiments, recombinant vaccinia viruses comprising one more mutation in the genome of the virus (virus back bone) such that the mutation increases the T-cell arm of the immune response. A mutation may be addition, deletion or substitution of one or more nucleic acid in the viral genome (wild type or attenuated native strains of vaccinia virus). In non-limiting examples, the mutation can be complete or partial deletion of genes that are known to inhibit cytokines involved in the Th1 immune response. As non-limiting examples the mutation may be deletion of nucleic acid encoding B8R (interferon gamma (IFN-g) binding proteins); C12L (interleukin-18 (IL-18) binding proteins). In further non-limiting example, the mutation can be complete or partial deletion of genes in innate immune signaling. As non-limiting examples the mutation may be deletion of nucleic acid encoding (B18R (type I interferon (IFN)-binding proteins); A52R (nuclear factor κB (NF-κB) inhibitor proteins); E3L (protein kinase (PKR) inhibitors); C4, C16 (STING pathway inhibitors). In further non-limiting example, the mutation can be complete or partial deletion of genes encoding proteins for inhibition of other components of the immune response. As non-limiting examples the mutation may be a complete or partial deletion of nucleic acid encoding B15, K7, B14, N1, K1, M2, A49, VH1, A46 or combination thereof. The viral backbone mutation may also include substituting the vaccinia virulence genes with genes of substantially equivalent function from other poxviruses. The vaccinia viruses provided herein comprise additional insertions, mutations, deletions or substitutions in the viral genome. A vaccinia virus may comprise one or more additional insertions or partial insertions of exogenous nucleic acids that code for one or more of chemokine receptor, TRIF protein or a functional domain thereof, or one or more of leptin, interleukin-2 (IL2), interleukin-15/interleukin-15Ra (IL15/IL15Ra), interleukin-7 (IL-7), leptin-interleukin fusion protein (e.g. leptin-IL2 fusion protein shown in example 1 as L2). Modifications such as insertion of chemokine receptor is insertion of wild type and/or mutant type CXCR4, CCR2, CCL2. A vaccinia virus may further comprise one or more additional deletions or partial deletions of one or more genes from A52R, B15R, K7R, A46R, N1L, E3L, K1L, M2L, C16, N2R, B8R, B18R, VH1 and a functional domain or fragment or variant thereof, or any combinations thereof. In some cases, the vaccinia virus provided herein can comprise a complete or partial deletion of the A52R gene and an insertion of a chemokine receptor, such as CCR2. In some cases, the vaccinia virus provided herein can comprise a complete or partial deletion of at least one of: A52R or TK viral genes, and insertion of an exogenous nucleic acid encoding a fusion protein (e.g., a metabolic modulator protein fused to a cytokine, such as Leptin-IL2 fusion protein).

### Additional Vectors

Vectors for delivery of nucleic acid constructs described herein can comprise physical, chemical, or biological means of transfection. In some embodiments, vectors provide for delivery of a protein described herein. In some embodiments, the vector is a lipid nanoparticle carrier.

In some embodiments, physical transfection comprises electroporation, thermal assisted gene transfer, biolistic (or gene gun), microinjection, laser-assisted transfection, ultrasound assisted gene transfer, hydrodynamic gene transfer, magnetofection, or mechanical massage, or any combination thereof. In some embodiments, the laser assisted transfection comprises optoinjection, laser influenced stress waves, photochemical internalization, or selective cell targeting via light absorbing particles. In some embodiments, microinjection comprises a single needle or an array.

In some embodiments, chemical transfection comprises calcium phosphate mediated transfection; diethylaminoethyl (DEAE) dextran mediated transfection; cationic lipid-mediated transfection; liposomes; polymers; other nanoparticles, such as gold, silica, carbon nanotubes, water soluble fullerenes, silicon nanowires, or quantum dots; or any combination thereof.

In some embodiments, transfection is mediated by cationic, ionizable, or other types of lipids. In some embodiments, lipids described herein comprise tetrakis(8-methylnonyl) 3,3′,3″,3″-(((methylazanediyl) bis(propane-3,1 diyl))bis (azanetriyl))tetrapropionate (3060mo); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); ((4-hydroxybutyl) azanediyl) bis(hexane-6,1-diyl)bis(2-hexyldecanoate) (ALC-0315); 2-[(polyethylene glycol)-2000]-N,N-ditetradecylacetamide (ALC-0159); (3S, 8S, 9S, 10R, 13R, 14S, 17R)-17-((2R, 5R)-5-ethyl-6-methylheptan-2-yl)-10,13-dimethyl-2, 3, 4, 7, 8, 9, 10, 11, 12, 13, 14, 15, 16, 17-tetradecahydro-1H-cyclopenta[a]phenanthren-3-ol (β-sitosterol); bis(2-(dodecyldisulfanyl)ethyl) 3, 3′-((3-methyl-9-oxo-10-oxa-13, 14-dithia-3, 6-diazahexacosyl)azanediyl)dipropionate (BAME-016B); 2-(((((3S, 8S, 9S, 10R, 13R, 14S, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl)oxy)carbonyl)amino)-N, N-bis(2-hydroxyethyl)-N-methylethan-1-aminium bromide (BHEM-Cholesterol): 1, 1′-((2-(4-(2-((2-(bis(2-hydroxydodecyl)amino)ethyl) (2-hydroxydodecyl)amino)ethyl) piperazin-1-yl)ethyl) azanediyl) bis(dodecan-2-ol) (C12-200); 3, 6-bis(4-(bis(2-hydroxydodecyl)amino) butyl)piperazine-2, 5-dione (cKK-E12); 3β-[N—(N′, N′-dimethylaminoethane)-carbamoyl]cholesterol (DC-Cholesterol); (6Z, 9Z, 28Z, 31Z)-heptatriaconta-6, 9, 28, 31-tetraen-19-yl 4-(dimethylamino) butanoate (DLin-MC3-DMA); 1, 2-dioleoyl-sn-glycero-3-phospho-ethanolamine (DOPE): 2, 3-dioleyloxy-N-[2-(sperminecarboxamido)ethyl]-N,N-dimethyl-1-propanaminium trifluoroacetate (DOSPA); 1,2-dioleoyl-3-trimethylammonium-propane (DOTAP); 1,2-di-O-octadecenyl-3-trimethylammonium-propane (DOTMA); 1,2-distearoyl-sn-glycero-3-phosphocholine; ePC, ethylphosphatidylcholine (DSPC); hexa(octan-3-yl) 9, 9′, 9″, 9″, 9″″, 7″-((((benzene-1,3,5-tricarbonyl)yris(azanediyl)) tris (propane-3,1-diyl)) tris(azanetriyl))hexanonanoate (FTT5); heptadecan-9-yl 8-((2-hydroxyethyl)(6-oxo-6-(undecyloxy)hexyl)amino) octanoate (Lipid H (SM-102)); (((3,6-dioxopiperazine-2,5-diyl)bis(butane-4, 1-diyl))bis(azanetriyl)) tetrakis(ethane-2,1-diyl) (9Z, 9′Z, 9″Z, 9″Z, 12Z, 12′Z, 12″Z, 12″Z)-tetrakis (octadeca-9,12-dienoate) (OF-Deg-Lin); 1,2-dimyristoyl-rac-glycero-3-methoxypolyethylene glycol-2000 (PEG2000-DMG,); N¹,N³,N⁵-tris(3-(didodecylamino)propyl)benzene-1, 3, 5-tricarboxamide (TT3), dioctadecyl-amidolysis-spermine, or any combination thereof.

In some embodiments, liposomes described herein are charged or neutral. In some embodiments, the liposomes are unilamellar or multilamellar vesicles. In some embodiments, the liposomes are inverted liposomes.

In some embodiments, polymers described herein comprise cationic peptides and derivatives thereof, such as polyomithine or polylysine. In some embodiments, cationic polymers comprise linear or branched synthetic polymers, including polyethyleneimine or polybrene. In some embodiments, cationic polymers comprise polysaccharide-based delivery compounds, including chitosan or cyclodextrin. In some embodiments, cationic polymers comprise natural polymers, including collagen or histone.

In some embodiments, carbon nanotubes (CNT) described herein comprise single-walled carbon nanotubes (SWNTs), multi-walled carbon nanotubes (MWNTs), or more. In some embodiments, CNTs described herein comprise concentric sheets rolled up as cylinders.

In some embodiments, quantum dots described herein comprise nanoparticles made of a semi-conducting material, such as cadmium, selenide, silicon, indium arsenide, admium sulphide, or any combination thereof.

In some embodiments, biological transfection comprises bacteriophage, virus-like particles (VLP), erythrocyte ghosts, bactofection, exosomes, or any combination thereof.

### Nucleic Acids

Provided herein are nucleic acids encoding for fusion constructs described herein. In some embodiments, the nucleic acids encode for fusion proteins. In some embodiments, the fusion proteins comprise full-length proteins, truncated versions of the full-length proteins, functional domains of the full-length proteins, fragments of the full-length proteins, variants of the full-length proteins, or any combination thereof. Variants of the full-length protein can comprise, in some examples, amino acid substitutions (conservative or non-conservative), deletions, additions, modifications, or any combinations thereof. In some embodiments, the nucleic acid encodes for a chemokine receptor. In some embodiments, the nucleic acid encodes for a trimerized fusion protein comprising a tumor necrosis super family member ligand (TNFSF-L). In some embodiments, the nucleic acid encodes for a Lymphotoxin alpha ligand, an OX40 ligand, a CD40 ligand, a Fas ligand, a CD27 ligand, CD30 ligand, CD137 ligand, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, a fragment thereof, or any combinations thereof.

Provided herein are nucleic acids encoding for fusion constructs comprising a TNFSF-L and an oligomerization domain. Exemplary TNFSF-Ls for inclusion include functional domains from Lymphotoxin alpha, OX40 ligand, CD40 ligand, Fas ligand, CD27 ligand, CD30 ligand, CD137 ligand, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof. Exemplary oligomerization domains include, without limitation, a collectin region from surfactant protein A (SP-A), surfactant protein D (SP-D), mannose binding lectin (MBL), conglutinin, CL-43, CL-L1, CL-K1, CL-P1, or CL-46. Sequences for exemplary fusion constructs including such a combination of features are provided in Table 4 (amino acid) and Table 5 (nucleic acid).

TABLE 4

| SEQ ID | | |
|---|---|---|
| NO: | Description | Amino Acid Sequence |
| 14 | 3sCD40L | MLLFLLSALVLLTQPLGYLEAEMKTYSHRIMPSACTLVMCSSVES |
| | (murine) | GLPGRDGSDVASLRQQVEALQGQVQHLQAAFSQYKKVEGSGHRRL |
| | | DKVEEEVNLHEDFVFIKKLKRCNKGEGSLSLLNCEEMRRQFEDLV |
| | | KDITLNKEEKKENSFEMQRGDEDPQIAAHVVSEANSNAASVLQWA |
| | | KKGYYTMKSNLVMLENGKQLTVKREGLYYVYTQVTFCSNREPSSQ |
| | | RPFIVGLWLKPSSGSERILLKAANTHSSSQLCEQQSVHLGGVFEL |
| | | QAGASVFVNVTEASQVIHRVGFSSFGLLKL* |
| 15 | 3sOX40L | MLLFLLSALVLLTQPLGYLEAEMKTYSHRIMPSACTLVMCSSVES |
| | (murine) | GLPGRDGSDVASLRQQVEALQGQVQHLQAAFSQYKKVESSSSPAK |
| | | DPPIQRLRGAVTRCEDGQLFISSYKNEYQTMEVQNNSVVIKCDGL |
| | | YIIYLKGSFFQEVKIDLHFREDHNPISIPMLNDGRRIVFTVVASL |
| | | AFKDKVYLTVNAPDTLCEHLQINDGELIVVQLTPGYCAPEGSYHS |
| | | TVNQVPL |
| 16 | 3s41BBL | MLLFLLSALVLLTQPLGYLEAEMKTYSHRIMPSACTLVMCSSVES |
| | (murine) | GLPGRDGSDVASLRQQVEALQGQVQHLQAAFSQYKKVESSHIGCP |
| | | NTTQQGSPVFAKLLAKNQASLCNTTLNWHSQDGAGSSYLSQGLRY |
| | | EEDKKELVVDSPGLYYVFLELKLSPTFTNTGHKVQGWVSLVLQAK |
| | | PQVDDFDNLALTVELFPCSMENKLVDRSWSQLLLLKAGHRLSVGL |
| | | RAYLHGAQDAYRDWELSYPNTTSFGLFLVKPDNPWE* |
| 17 | 3sGITRL | MLLFLLSALVLLTQPLGYLEAEMKTYSHRIMPSACTLVMCSSVES |
| | (murine) | GLPGRDGSDVASLRQQVEALQGQVQHLQAAFSQYKKVEGSGTSLK |
| | | PTAIESCMVKFELSSSKWHMTSPKPHCVNTTSDGKLKILQSGTYL |
| | | IYGQVIPVDKKYIKDNAPFVVQIYKKNDVLQTLMNDFQILPIGGV |
| | | YELHAGDNIYLKFNSKDHIQKTNTYWGIILMPDLPFIS |
| 18 | 3sOX40L | MLLFLLSALVLLTQPLGYLEAEMKTYSHRIMPSACTLVMCSSVES |
| | (human) | GLPGRDGSDVASLRQQVEALQGQVQHLQAAFSQYKKVEGSGRIQS |
| | | IKVQFTEYKKEKGFILTSQKEDEIMKVQNNSVIINCDGFYLISLK |
| | | GYFSQEVNISLHYQKDEEPLFQLKKVRSVNSLMVASLTYKDKVYL |
| | | NVTTDNTSLDDFHVNGGELILIHQNPGEFCVL |
| 19 | 3s41BBL | MLLFLLSALVLLTQPLGYLEAEMKTYSHRIMPSACTLVMCSSVES |
| | (human) | GLPGRDGSDVASLRQQVEALQGQVQHLQAAFSQYKKVEGSGQGMF |
| | | AQLVAQNVLLIDGPLSWYSDPGLAGVSLTGGLSYKEDTKELVVAK |
| | | AGVYYVFFQLELRRVVAGEGSGSVSLALHLQPLRSAAGAAALALT |
| | | VDLPPASSEARNSAFGFQGRLLHLSAGQRLGVHLHTEARARHAWQ |
| | | LTQGATVLGLFRVTPEIPAGLPSPRSE* |
| 20 | 3sGITRL | MLLFLLSALVLLTQPLGYLEAEMKTYSHRIMPSACTLVMCSSVES |
| | (human) | GLPGRDGSDVASLRQQVEALQGQVQHLQAAFSQYKKVEGSGTAKE |
| | | PCMAKFGPLPSKWQMASSEPPCVNKVSDWKLEILQNGLYLIYGQV |
| | | APNANYNDVAPFEVRLYKNKDMIQTLTNKSKIQNVGGTYELHVGD |
| | | TIDLIFNSEHQVLKNNTYWGIILLANPQFIS |

TABLE 5

| SEQ ID | | |
|---|---|---|
| NO: | Description | Nucleic Acid Sequence |
| 21 | 3sCD40L | ATGCTGCTCTTCCTCCTCTCTGCACTGGTCCTGCTCACACAGCC |
| | (murine) | CCTGGGCTACCTGGAAGCAGAAATGAAGACCTACTCCCACAGAA |
| | | CAATGCCCAGTGCTTGCACCCTGGTCATGTGTAGCTCAGTGGAG |
| | | AGTGGCCTGCCTGGTCGCGATGGATCTGATGTTGCTTCTCTGAG |
| | | GCAGCAGGTTGAGGCCTTACAGGGACAAGTACAGCACCTCCAGG |
| | | CTGCTTTCTCTCAGTATAAGAAAGTGGAGGGATCCGGTCACAGG |
| | | CGTCTGGACAAGGTCGAAGAGGAAGTAAACCTTCATGAAGATTT |
| | | TGTATTCATAAAAAAGCTAAAGAGATGCAACAAAGGAGAAGGAT |
| | | CTTTATCCTTGCTGAACTGTGAGGAGATGAGAAGGCAATTTGAA |
| | | GACCTTGTCAAGGATATAACGTTAAACAAAGAAGAGAAAAAAGA |
| | | AAACAGCTTTGAAATGCAAAGAGGTGATGAGGATCCTCAAATTG |
| | | CAGCACACGTTGTAAGCGAAGCCAACAGTAATGCAGCATCCGTT |
| | | CTACAGTGGGCCAAGAAAGGATATTATACCATGAAAAGCAACTT |
| | | GGTAATGCTTGAAAATGGGAAACAGCTGACGGTTAAAAGAGAAG |
| | | GACTCTATTATGTCTACACTCAAGTCACCTTCTGCTCTAATCGG |
| | | GAGCCTTCGAGTCAACGCCCATTCATCGTCGGCCTCTGGCTGAA |
| | | GCCCAGCAGTGGATCTGAGAGAATCTTACTCAAGGCGGCAAATA |
| | | CCCACAGTTCCTCCCAGCTTTGCGAGCAGCAGTCTGTTCACTTG |
| | | GGCGGAGTGTTTGAATTACAAGCTGGTGCTTCTGTGTTTGTCAA |
| | | CGTGACTGAAGCAAGCCAAGTGATCCACAGAGTTGGCTTCTCAT |
| | | CTTTTGGCTTACTCAAACTCTGA |
| 22 | 3sOX40L | ATGCTGCTCTTCCTCCTCTCTGCACTGGTCCTGCTCACACAGCC |
| | (murine) | CCTGGGCTACCTGGAAGCAGAAATGAAGACCTACTCCCACAGAA |
| | | CAATGCCCAGTGCTTGCACCCTGGTCATGTGTAGCTCAGTGGAG |
| | | AGcGGCCTGCCTGGTCGCGATGGATCTGATGTTGCTTCTCTGAG |
| | | GCAGCAGGTTGAGGCCTTACAGGGACAAGTACAGCACCTCCAGG |
| | | CTGCTTTCTCTCAGTATAAGAAAGTAGAATCATCTTCCTCTCCG |
| | | GCAAAGGACCCTCCAATCCAAAGACTCAGAGGAGCAGTTACCAG |
| | | ATGTGAGGATGGGCAACTATTCATCAGCTCATACAAGAATGAGT |
| | | ATCAAACTATGGAGGTGCAGAACAATTCGGTTGTCATCAAGTGC |
| | | GATGGGCTTTATATCATCTACCTGAAGGGCTCCTTTTTCCAGGA |
| | | GGTCAAGATTGACCTTCATTTCCGGGAGGATCATAATCCCATCT |
| | | CTATTCCAATGCTGAACGATGGTCGAAGGATTGTCTTCACTGTG |
| | | GTGGCCTCTTTGGCTTTCAAAGATAAAGTTTACCTGACTGTAAA |
| | | TGCTCCTGATACTCTCTGCGAACACCTCCAGATAAATGATGGGG |
| | | AGCTGATTGTTGTCCAGCTAACGCCTGGATACTGTGCTCCTGAA |
| | | GGATCTTACCACAGCACTGTGAACCAAGTACCACTGTGA |
| 23 | 3s41BBL | ATGCTGCTCTTCCTCCTCTCTGCACTGGTCCTGCTCACACAGCC |
| | (murine) | CCTGGGCTACCTGGAAGCAGAAATGAAGACCTACTCCCACAGAA |
| | | CAATGCCCAGTGCTTGCACCCTGGTCATGTGTAGCTCAGTGGAG |
| | | AGTGGCCTGCCTGGTCGCGATGGATCTGATGTTGCTTCTCTGAG |
| | | GCAGCAGGTTGAGGCCTTACAGGGACAAGTACAGCACCTCCAGG |
| | | CTGCTTTCTCTCAGTATAAGAAAGTAGAATCATCCCACATTGGC |
| | | TGCCCCAACACTACACAACAGGGCTCTCCTGTGTTCGCCAAGCT |
| | | ACTGGCTAAAAACCAAGCATCGTTGTGCAATACAACTCTGAACT |
| | | GGCACAGCCAAGATGGAGCTGGGtctTCATACCTATCTCAAGGT |
| | | CTGAGGTACGAAGAAGACAAAAAGGAGTTGGTGGTAGACAGTCC |
| | | CGGGCTCTACTACGTATTTTTGGAACTGAAGCTCAGTCCAACAT |
| | | TCACAAACACAGGCCACAAGGTGCAGGGCTGGGTCTCTCTTGTT |
| | | TTGCAAGCAAAGCCTCAGGTAGATGACTTTGACAACTTGGCCCT |
| | | GACAGTGGAACTGTTCCCTTGCTCCATGGAGAACAAGTTAGTGG |
| | | ACCGTTCCTGGAGTCAACTGTTGCTCCTGAAGGCTGGCCACCGC |
| | | CTCAGTGTGGGTCTGAGGGCTTATCTGCATGGAGCCCAGGATGC |
| | | ATACAGAGACTGGGAGCTGTCTTATCCCAACACCACCAGCTTTG |
| | | GACTCTTTCTTGTGAAACCCGACAACCCATGGGAATGA |
| 24 | 3xGITRL | ATGCTGCTCTTCCTCCTCTCTGCACTGGTCCTGCTCACACAGCC |
| | (murine) | CCTGGGCTACCTGGAAGCAGAAATGAAGACCTACTCCCACAGAA |
| | | CAATGCCCAGTGCTTGCACCCTGGTCATGTGTAGCTCAGTGGAG |
| | | AGTGGCCTGCCTGGTCGCGATGGATCTGATGTTGCTTCTCTGAG |
| | | GCAGCAGGTTGAGGCCTTACAGGGACAAGTACAGCACCTCCAGG |
| | | CTGCTTTCTCTCAGTATAAGAAAGTGGAGGGATCCGGTACTTCA |
| | | CTCAAGCCAACTGCCATCGAGTCCTGCATGGTTAAGTTTGAACT |
| | | ATCATCCTCAAAATGGCACATGACATCTCCCAAACCTCACTGTG |
| | | TGAATACGACATCTGATGGGAAGCTGAAGATACTGCAGAGTGGC |
| | | ACATATTTAATCTACGGCCAAGTGATTCCTGTGGATAAGAAATA |
| | | CATAAAAGACAATGCCCCCTTCGTAGTACAGATATATAAAAAGA |
| | | ATGATGTCCTACAAACTCTAATGAATGATTTTCAAATCTTGCCT |
| | | ATAGGAGGGGTTTATGAACTGCATGCTGGAGATAACATATATCT |
| | | GAAGTTCAACTCTAAAGACCATATTCAGAAAACTAACACATACT |
| | | GGGGGATCATCTTAATGCCTGATCTACCATTCATCTCTTAG |
| 25 | 3sOX40L | ATGCTGCTCTTCCTCCTCTCTGCACTGGTCCTGCTCACACAGCC |
| | (human) | CCTGGGCTACCTGGAAGCAGAAATGAAGACCTACTCCCACAGAA |
| | | CAATGCCCAGTGCTTGCACCCTGGTCATGTGTAGCTCAGTGGAG |
| | | AGTGGCCTGCCTGGTCGCGATGGATCTGATGTTGCTTCTCTGAG |
| | | GCAGCAGGTTGAGGCCTTACAGGGACAAGTACAGCACCTCCAGG |
| | | CTGCTTTCTCTCAGTATAAGAAAGTGGAGGGATCCGGTCGAATT |
| | | CAAAGTATCAAAGTACAATTTACCGAATATAAGAAGGAGAAAGG |
| | | TTTCATCCTCACTTCCCAAAAGGAGGATGAAATCATGAAGGTGC |
| | | AGAACAACTCAGTCATCATCAACTGTGATGGGTTTTATCTCATC |
| | | TCCCTGAAGGGCTACTTCTCCCAGGAAGTCAACATTAGCCTTCA |
| | | TTACCAGAAGGATGAGGAGCCCCTCTTCCAACTGAAGAAGGTCA |
| | | GGTCTGTCAACTCCTTGATGGTGGCCTCTCTGACTTACAAAGAC |
| | | AAAGTCTACTTGAATGTGACCACTGACAATACCTCCCTGGATGA |
| | | CTTCCATGTGAATGGCGGAGAACTGATTCTTATCCATCAAAATC |
| | | CTGGTGAATTCTGTGTCCTTTGA |
| 26 | 3s41BBL | ATGCTGCTCTTCCTCCTCTCTGCACTGGTCCTGCTCACACAGCC |
| | (human) | CCTGGGCTACCTGGAAGCAGAAATGAAGACCTACTCCCACAGAA |
| | | CAATGCCCAGTGCTTGCACCCTGGTCATGTGTAGCTCAGTGGAG |
| | | AGTGGCCTGCCTGGTCGCGATGGATCTGATGTTGCTTCTCTGAG |
| | | GCAGCAGGTTGAGGCCTTACAGGGACAAGTACAGCACCTCCAGG |
| | | CTGCTTTCTCTCAGTATAAGAAAGTGTGTTCTGCTGATCGATGG |
| | | GCCCCTGAGCTGGTACAGTGACCCAGGCCTGGCAGGCGTGTCCC |
| | | TGACGGGGGGCCTGAGCTACAAAGAGGACACGAAGGAGCTGGTG |
| | | GTGGCCAAGGCTGGAGTCTACTATGTCTTCTTTCAACTAGAGCT |
| | | GCGGCGCGTGGTGGCCGGCGAGGGCTCAGGCTCCGTTTCACTTG |
| | | CGCTGCACCTGCAGCCACTGCGCTCTGCTGCTGGGGCCGCCGCC |
| | | CTGGCTTTGACCGTGGACCTGCCACCCGCCTCCTCCGAGGCTCG |
| | | GAACTCGGCCTTCGGTTTCCAGGGCCGCTTGCTGCACCTGAGTG |
| | | CCGGCCAGCGCCTGGGCGTCCATCTTCACACTGAGGCCAGGGCA |
| | | CGCCATGCCTGGCAGCTTACCCAGGGCGCCACAGTCTTGGGACT |
| | | CTTCCGGGTGACCCCCGAAATCCCAGCCGGACTCCCTTCACCGA |
| | | GGTCGGAATAA |
| 27 | 3sGITRL | ATGCTGCTCTTCCTCCTCTCTGCACTGGTCCTGCTCACACAGCC |
| | (human) | CCTGGGCTACCTGGAAGCAGAAATGAAGACCTACTCCCACAGAA |
| | | CAATGCCCAGTGCTTGCACCCTGGTCATGTGTAGCTCAGTGGAG |
| | | AGTGGCCTGCCTGGTCGCGATGGATCTGATGTTGCTTCTCTGAG |
| | | GCAGCAGGTTGAGGCCTTACAGGGACAAGTACAGCACCTCCAGG |
| | | CTGCTTTCTCTCAGTATAAGAAAGTGGAGGGATCCGGTACTGCT |
| | | AAGGAGCCCTGTATGGCTAAGTTTGGACCATTACCCTCAAAATG |
| | | GCAAATGGCATCTTCTGAACCTCCTTGCGTGAATAAGGTGTCTG |
| | | ACTGGAAGCTGGAGATACTTCAGAATGGCTTATATTTAATTTAT |
| | | GGCCAAGTGGCTCCCAATGCAAACTACAATGATGTAGCTCCTTT |
| | | TGAGGTGCGGCTGTATAAAAACAAAGACATGATACAAACTCTAA |
| | | CAAACAAATCTAAAATCCAAAATGTAGGAGGGACTTATGAATTG |
| | | CATGTTGGGGACACCATAGACTTGATATTCAACTCTGAGCATCA |
| | | GGTTCTAAAAAATAATACATACTGGGGTATCATTTTACTAGCAA |
| | | ATCCCCAATTCATCTCCTAG |

In some embodiments, the nucleic acids described herein are combined with a vector. In some embodiments, the nucleic acids are incorporated into a genome. In some embodiments, the genome is a viral genome, a bacterial genome, or a genome of a cell. In some embodiments, the cell is a mammalian cell. In some embodiments, the mammalian cell is an immune cell. In some embodiments, the mammalian cell is a tumor cell.

Provided here are modified oncolytic viruses comprising one or more of exogenous nucleic acids that code for fusion proteins, wherein the fusion proteins comprise the fill-length proteins, truncated versions of the fill-length proteins, functional domains of the full-length proteins, fragments of the full-length proteins, variants of the full-length proteins, or any combination thereof. Variants of the full-length protein can comprise, in some examples, amino acid substitutions (conservative or non-conservative), deletions, additions, modifications, or any combinations thereof. In some embodiments, provided herein is a modified oncolytic virus comprising an exogenous nucleic acid, also referred to herein as a transgene, that can code for a chemokine receptor. In some cases, the exogenous nucleic acid can be a therapeutic transgene. In some embodiments, provided herein is a modified oncolytic virus comprising an exogenous nucleic acid that can code for a trimerized fusion protein comprising a tumor necrosis super family member ligand (TNFSF-L).

In some embodiments, the modified oncolytic virus comprises an exogenous nucleic acid that codes for a Lymphotoxin alpha ligand, an OX40 ligand, a CD40 ligand, a Fas ligand, a CD27 ligand, CD30 ligand, CD137 ligand, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, a fragment thereof, or any combinations thereof. Each or any combinations of these proteins may contribute to a greater therapeutic benefit of the backbone oncolytic virus.

In some embodiments, the modification of the oncolytic virus can result in at least about 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 1×10⁴, 2.5×10⁴, 5×10⁴, 7.5×10⁴, 1×10⁵, 2.5×10⁵, 5×10⁵, 7.5×10⁵, 1×10⁶, 2.5×10⁶, 5×10⁶, 7.5×10⁶, 1×10⁷, 2.5×10⁷, 5×10⁷, 7.5×10⁷, 1×10⁸, 2.5×10⁸, 5×10⁸, 7.5×10⁸, 1×10⁹, 2.5×10⁹, 5×10⁹, 7.5×10⁹, 1×10¹⁰ or even more folds increase in the efficacy of tumor-targeted systemic delivery of the virus, as compared to an otherwise identical oncolytic virus that does not comprise the modification. In some embodiments, the efficacy of tumor-targeted systemic delivery of the virus can be measured by quantifying the viruses infecting the tumor cells, and optionally, in contrast with the viruses infecting non-tumor cells in the body. In some embodiments, the quantification of the virus can be performed by staining the viral particles in tissue sections, or blood smear in the cases of leukemia, lymphoma, or myeloma. In some embodiments, quantification can be performed by reporter molecule(s) that is/are engineered to be expressed by the viruses, e.g., luciferase, and fluorescent proteins. In some cases, such quantification can be performed by quantifying the viral genome in the tumor. Without being limited, the tumor-targeted systemic delivery of the virus can be measured by quantifying certain downstream effect(s) of viral infection in tumor cells, for example, cytokines in response to viral infection or lymphocyte accumulation. In some embodiments, the oncolytic virus comprises an exogenous nucleic acid that can code for a oligomerized fusion protein designed for extracellular release comprising a tumor necrosis super family member ligand (TNFSF-L) region, and the presence of the exogenous nucleic acid can result in about 5 to 10 folds increase in the efficacy of tumor-target systemic delivery of the virus, as compared to an otherwise identical oncolytic virus that does not comprise the exogenous nucleic acid.

In some embodiments, a modified oncolytic virus comprises an exogenous nucleic acid that encodes for a trimerized fusion protein designed for extracellular release comprising a TNFSF-L region and an oligomerization region, and the expression of this fusion protein by the modified oncolytic virus can result in boosted immune responses against the infected tumor. Consequently, the immunosuppressive microenvironment in the tumor can be altered, leading to enhanced immunotherapeutic activity of the modified oncolytic virus, as compared to an otherwise identical virus that does comprise the nucleic acid coding for the aforementioned fusion protein. In some embodiments, the increase in immunotherapeutic activity can be at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 1×10⁴, 2.5×10⁴, 5×10⁴, 7.5×10⁴, 2.5×10⁵, 5×10⁵, 1×10⁶ or even higher folds. Without being limited, the increased immunotherapeutic activity can be reflected by increased B cell accumulation in the tumor, increased T cell response to tumor-related immunogens, or both. B cell accumulation can be measured, for example, by quantifying the B cells in the tumor, and T cell immuno-activity may be measured by, for example, interferon-γ (interferon-gamma) secretion in ELISPOT assays.

Provided herein are oncolytic viruses comprising an exogenous nucleic acid sequence encoding fusion constructs comprising a TNFSF-L and an oligomerization domain. Exemplary TNFSF-Ls for inclusion include functional domains from Lymphotoxin alpha, OX40 ligand, CD40 ligand, Fas ligand, CD27 ligand, CD30 ligand, CD137 ligand, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, any combination thereof. Exemplary oligomerization domains include, without limitation, a collectin region from surfactant protein A (SP-A), surfactant protein D (SP-D), mannose binding lectin (MBL), conglutinin, CL-43, CL-L1, CL-K1, CL-P1, or CL-46. Exemplary fusion constructs including such a combination of features are provided in Table 4 (amino acid) and Table 5 (nucleic acid).

### Conditions and Administration

Provided herein are fusion constructs for use in an expression system for treatment of cancer. In some embodiments, the fusion constructs are expressed within a cancer cell. In some embodiments, the cancer is a blood cancer (aka hematopoietic cancer) or a solid cancer. In some embodiments, the cancer includes, without limitation, melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, and sarcoma. In some embodiments, the cancer is a cancer of the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus.

In some embodiments, the fusion construct is expressed in an immune cell. In some embodiments, the cell is a lymphoid or myeloid cell. In some embodiments, the lymphoid cell is a B cell, a Natural Killer cell, or a T cell. In some embodiments, the T cell is an effector cell. In some embodiments, the effector cell is a cytotoxic T cell or CD8+ cell. In some embodiments, the effector cell is a helper cell or CD4+ cell. In some embodiments, the effector cell is a regulatory T cell. In some embodiments the T cell is a memory T cell. In some embodiments, the myeloid cell is a neutrophil, an eosinophil, or a monocyte.

Provided herein are fusion constructs for use in an expression system for reduction of tumor cell growth. In some embodiments, the tumor is a liquid tumor or a solid tumor. In some embodiments, the tumor comprises a melanoma, a hepatocellular carcinoma, a breast tumor, a lung tumor, a peritoneal tumor, a prostate tumor, a bladder tumor, an ovarian tumor, a leukemia, a lymphoma, a renal carcinoma, a pancreatic tumor, an epithelial carcinoma, a gastric tumor, a colon carcinoma, a duodenal tumor, a pancreatic adenocarcinoma, a mesothelioma, a glioblastoma multiforme, an astrocytoma, a multiple myeloma, a prostate carcinoma, a hepatocellular carcinoma, a cholangiosarcoma, a pancreatic adenocarcinoma, a head and neck squamous cell carcinoma, a colorectal tumor, an intestinal-type gastric adenocarcinoma, a cervical squamous-cell carcinoma, an osteosarcoma, an epithelial ovarian carcinoma, an acute lymphoblastic lymphoma, a myeloproliferative neoplasm, or a sarcoma. In some embodiments, the tumor comprises a tumor of the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus.

In some embodiments, this disclosure provides methods for treating a subject by administration of one or more modified oncolytic viruses, as disclosed herein. An “individual” or “subject”: as used interchangeably herein, refers to a human or a non-human subject. Non-limiting examples of non-human subjects include non-human primates, dogs, cats, mice, rats, guinea pigs, rabbits, pigs, fowl, horses, cows, goats, sheep, cetaceans, etc. In some embodiments, the subject is human.

In some embodiments, provided herein is a method of producing a toxic effect in a cancer cell comprising administering, to the cancer cell, a therapeutically effective amount of a modified virus, such as an oncolytic vaccinia virus, as described above, or a pharmaceutical composition comprising the same. This disclosure further provides a method of inhibiting at least one of growth and proliferation of a second cancer cell comprising administering, to a first cancer cell, a modified oncolytic virus as described above such that the first cancer cell is infected with said virus. Thus, in some embodiments of the methods disclosed here, it is contemplated that not every cancer or tumor cell is infected upon administering a therapeutically effective amount of an oncolytic vaccinia virus, as described herein, or a pharmaceutical composition comprising the same, and growth of non-infected cells can be inhibited without direct infection.

In some embodiments, to induce oncolysis, kill cells, inhibit growth, inhibit metastases, decrease tumor size and otherwise reverse or reduce the malignant phenotype of tumor cells, using the methods and compositions of the present disclosure, a cancer cell or a tumor can be contacted with a therapeutically effective dose of an exemplary oncolytic vaccinia virus as described herein or a pharmaceutical composition comprising the same. In some embodiments, an effective amount of a modified oncolytic virus of the present disclosure, such as an oncolytic vaccinia virus as described herein or a pharmaceutical composition thereof, can include an amount sufficient to induce oncolysis, the disruption or lysis of a cancer cell or the inhibition or reduction in the growth or size of a cancer cell. Reducing the growth of a cancer cell may be manifested, for example, by cell death or a slower replication rate or reduced growth rate of a tumor comprising the cell or a prolonged survival of a subject comprising the cancer cell.

Provided, in some embodiments, is a method of treating a subject having a cancer or a tumor comprising administering, to the subject, an effective amount of a nucleic acid, fusion protein, or modified virus, as described above. An effective amount in such method can include an amount that reduces growth rate or spread of the cancer or that prolongs survival in the subject. This disclosure provides a method of reducing the growth of a tumor, which method can comprise administering, to the tumor, an effective amount of a nucleic acid, fusion protein, or modified oncolytic virus as described above. In some embodiments, an effective amount of a nucleic acid, fusion protein, or modified virus, or a pharmaceutical composition thereof, can include an amount sufficient to induce the slowing, inhibition or reduction in the growth or size of a tumor and can include the eradication of the tumor. Reducing the growth of a tumor may be manifested, for example, by reduced growth rate or a prolonged survival of a subject comprising the tumor.

In some embodiments, a nucleic acid as described herein is administered to a subject in an amount from about 0.01 μg/dose to about 1 g/dose or from about 0.5 μg/dose to about 500 mg/dose. In some embodiments, the nucleic acid is administered to a subject at about 0.01 μg/dose, about 0.05 μg/dose, about 0.1 μg/dose, about 0.5 μg/dose, about 1 μg/dose, about 5 μg/dose, about 10 μg/dose, about 50 μg/dose, about 100 μg/dose, about 500 μg/dose, about 1 mg/dose, about 5 mg/dose, about 10 mg/dose, about 50 mg/dose, about 100 mg/dose, about 500 mg/dose, about 1 g/dose.

In some embodiments, cells as described herein is administered to a subject in an amount from about 0.01 μg/dose to about 1 g/dose or from about 0.5 μg/dose to about 500 mg/dose. In some embodiments, the cells are administered to a subject at about 0.01 μg/dose, about 0.05 μg/dose, about 0.1 μg/dose, about 0.5 μg/dose, about 1 μg/dose, about 5 μg/dose, about 10 μg/dose, about 50 μg/dose, about 100 μg/dose, about 500 μg/dose, about 1 mg/dose, about 5 mg/dose, about 10 mg/dose, about 50 mg/dose, about 100 mg/dose, about 500 mg/dose, about 1 g/dose.

In some embodiments, a fusion protein as described herein is administered to a subject in an amount from about 0.01 μg/dose to about 1 g/dose or from about 0.5 μg/dose to about 500 mg/dose. In some embodiments, the fusion protein is administered to a subject at about 0.01 μg/dose, about 0.05 μg/dose, about 0.1 μg/dose, about 0.5 μg/dose, about 1 μg/dose, about 5 μg/dose, about 10 μg/dose, about 50 μg/dose, about 100 μg/dose, about 500 μg/dose, about 1 mg/dose, about 5 mg/dose, about 10 mg/dose, about 50 mg/dose, about 100 mg/dose, about 500 mg/dose, about 1 g/dose.

In some embodiments, an amount of a modified oncolytic virus of this disclosure, such as an oncolytic virus or vaccinia virus, administered to a subject is between about 10³ and 10¹² infectious viral particles or plaque forming units (PFU), or between about 10⁵ and 10¹⁰ PFU, or between about 10⁵ and 10⁸ PFU, or between about 10⁸ and 10¹⁰ PFU. In some embodiments, the amount of a modified oncolytic virus of this disclosure, such as an oncolytic virus or vaccinia virus administered to a subject is between about 10³ and 10¹² viral particles or plaque forming units (PFU), or between about 10⁵ and 10¹⁰ PFU, or between about 10⁵ and 10⁸ PFU, or between about 10⁸ and 10¹⁰ PFU. In some embodiments, a modified oncolytic virus of this disclosure, such as an oncolytic vaccinia virus, is administered at a dose that can comprise about 10³ PFU/dose to about 10⁴ PFU/dose, about 10⁴ PFU/dose to about 10⁵ PFU/dose, about 10⁵ PFU/dose to about 10⁶ PFU/dose, about 10⁷ PFU/dose to about 10⁸ PFU/dose, about 10⁹ PFU/dose to about 10¹⁰ PFU/dose, about 10¹⁰ PFU/dose to about 10¹¹ PFU/dose, about 10¹¹ PFU/dose to about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic virus of this disclosure, such as an oncolytic vaccinia virus, can be administered at a dose that can comprise about 2×10³ PFU/dose, 3×10³ PFU/dose, 4×10³ PFU/dose, 5×10³ PFU/dose, 6×10³ PFU/dose, 7×10³ PFU/dose, 8×10³ PFU/dose, 9×10³ PFU/dose, about 10⁴ PFU/dose, about 2×10⁴ PFU/dose, about 3×10⁴ PFU/dose, about 4×10⁴ PFU/dose, about 5×10⁴ PFU/dose, about 6×10⁴ PFU/dose, about 7×10⁴ PFU/dose, about 8×10⁴ PFU/dose, about 9×10⁴ PFU/dose, about 10⁵ PFU/dose, 2×10⁵ PFU/dose, 3×10⁵ PFU/dose, 4×10⁵ PFU/dose 5×10⁵ PFU/dose, 6×10⁵ PFU/dose, 7×10⁵ PFU/dose, 8×10⁵ PFU/dose, 9×10⁵ PFU/dose about 10⁶ PFU/dose, about 2×10⁶ PFU/dose, about 3×10⁶ PFU/dose, about 4×10⁶ PFU/dose about 5×10⁶ PFU/dose, about 6×10⁶ PFU/dose, about 7×10⁶ PFU/dose, about 8×10⁶ PFU/dose, about 9×10⁶ PFU/dose, about 10⁷ PFU/dose, about 2×10⁷ PFU/dose, about 3×10⁷ PFU/dose, about 4×10⁷ PFU/dose, about 5×10⁷ PFU/dose, about 6×10⁷ PFU/dose, about 7×10⁷ PFU/dose, about 8×10⁷ PFU/dose, about 9×10⁷ PFU/dose, about 10⁸ PFU/dose, about 2×10⁸ PFU/dose, about 3×10⁸ PFU/dose, about 4×10⁸ PFU/dose, about 5×10⁸ PFU/dose, about 6×10⁸ PFU/dose, about 7×10⁸ PFU/dose, about 8×10⁸ PFU/dose, about 9×10⁸ PFU/dose, about 10⁹ PFU/dose, about 2×10⁹ PFU/dose, about 3×10⁹ PFU/dose, about 4×10⁹ PFU/dose, about 5×10⁹ PFU/dose, about 6×10⁹ PFU/dose, about 7×10⁹ PFU/dose, about 8×10⁹ PFU/dose, about 9×10⁹ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹¹ PFU/dose, about 2×10¹¹ PFU/dose, about 3×10¹¹ PFU/dose, about 4×10¹¹ PFU/dose, about 5×10¹¹ PFU/dose, about 6×10¹¹ PFU/dose, about 7×10¹¹ PFU/dose, about 8×10¹¹ PFU/dose, about 9×10¹¹ PFU/dose, or about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic virus of this disclosure, such as an oncolytic vaccinia virus, can be administered at a dose that can comprise 5×10⁹ PFU/dose. In some embodiments, a modified oncolytic virus of this disclosure, such as an oncolytic vaccinia virus, can be administered at a dose that can comprise up to 5×10⁹ PFU/dose.

In some embodiments, a modified oncolytic virus of this disclosure, such as an oncolytic vaccinia virus, can be administered at a dose that can comprise about 10³ viral particles/dose to about 10⁴ viral particles/dose, about 10⁴ viral particles/dose to about 10⁵ viral particles/dose, about 10⁵ viral particles/dose to about 10⁶ viral particles/dose, about 10⁷ viral particles/dose to about 10⁸ viral particles/dose, about 10⁹ viral particles/dose to about 10¹⁰ viral particles/dose, about 10¹⁰ viral particles/dose to about 10¹¹ viral particles/dose, about 10¹¹ viral particles/dose to about 10¹² viral particles/dose, about 10¹²viral particles/dose to about 10¹³ viral particles/dose, about 10¹³ viral particles/dose to about 10¹⁴ viral particles/dose, or about 10¹⁴ viral particles/dose to about 10¹⁵ viral particles/dose.

In some embodiments, a modified oncolytic virus of this disclosure can be administered at a dose that can comprise about 10³ PFU/kg to about 10⁴ PFU/kg, about 10⁴ PFU/kg to about 10⁵ PFU/kg, about 10⁵ PFU/kg to about 10⁶ PFU/kg, about 10⁷ PFU/kg to about 10⁸ PFU/kg, about 10⁹ PFU/kg to about 10¹⁰ PFU/kg, about 10¹⁰ PFU/kg to about 10¹¹ PFU/kg, about 10¹¹ PFU/kg to about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic virus of this disclosure, such as an oncolytic vaccinia virus, can be administered at a dose that can comprise about 2×10³ PFU/kg, 3×10³ PFU/kg, 4×10³ PFU/kg, 5×10³ PFU/kg, 6×10³ PFU/kg, 7×10³ PFU/kg, 8×10³ PFU/kg, 9×10³ PFU/kg, about 10⁴ PFU/kg, about 2×10⁴ PFU/kg, about 3×10⁴ PFU/kg, about 4×10⁴ PFU/kg, about 5×10⁴ PFU/kg, about 6×10⁴ PFU/kg, about 7×10⁴ PFU/kg, about 8×10⁴ PFU/kg, about 9×10⁴ PFU/kg, about 10⁵ PFU/kg, 2×10⁵ PFU/kg, 3×10⁵ PFU/kg, 4×10⁵ PFU/kg, 5×10⁵ PFU/kg, 6×10⁵ PFU/kg, 7×10⁵ PFU/kg, 8×10⁵ PFU/kg, 9×10⁵ PFU/kg, about 10⁶ PFU/kg, about 2×10⁶PFU/kg, about 3×10⁶ PFU/kg, about 4×10⁶ PFU/kg, about 5×10⁶ PFU/kg, about 6×10⁶ PFU/kg, about 7×10⁶ PFU/kg, about 8×10⁶ PFU/kg, about 9×10⁶ PFU/kg, about 10⁷ PFU/kg, about 2×10⁷ PFU/kg, about 3×10⁷ PFU/kg, about 4×10⁷ PFU/kg, about 5×10⁷ PFU/kg, about 6×10⁷ PFU/kg, about 7×10⁷ PFU/kg, about 8×10⁷ PFU/kg, about 9×10⁷ PFU/kg, about 10⁸ PFU/kg, about 2×10⁸ PFU/kg, about 3×10⁸ PFU/kg, about 4×10⁸ PFU/kg, about 5×10⁸ PFU/kg, about 6×10⁸ PFU/kg, about 7×10⁸ PFU/kg, about 8×10⁸ PFU/kg, about 9×10⁸ PFU/kg, about 10⁹ PFU/kg, about 2×10⁹ PFU/kg, about 3×10⁹ PFU/kg, about 4×10⁹ PFU/kg, about 5×10⁹ PFU/kg, about 6×10⁹ PFU/kg, about 7×10⁹ PFU/kg, about 8×10⁹ PFU/kg, about 9×10⁹ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹¹ PFU/kg, about 2×10¹¹ PFU/kg, about 3×10¹¹ PFU/kg, about 4×10¹¹ PFU/kg, about 5×10¹¹ PFU/kg, about 6×10¹¹ PFU/kg, about 7×10¹¹ PFU/kg, about 8×10¹¹ PFU/kg, about 9×10¹¹ PFU/kg, or about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic virus of this disclosure, such as an oncolytic vaccinia virus, can be administered at a dose that can comprise 5×10⁹ PFU/kg. In some embodiments, a modified oncolytic virus of this disclosure, such as an oncolytic vaccinia virus, can be administered at a dose that can comprise up to 5×10⁹ PFU/kg.

In some embodiments, a modified oncolytic virus of this disclosure can be administered at a dose that can comprise about 10³ viral particles/kg to about 10⁴ viral particles/kg, about 10⁴ viral particles/kg to about 10⁵ viral particles/kg, about 10⁵ viral particles/kg to about 10⁶ viral particles/kg, about 10⁷ viral particles/kg to about 10⁸ viral particles/kg, about 10⁹ viral particles/kg to about 10¹⁰ viral particles/kg, about 10¹⁰ viral particles/kg to about 10¹¹ viral particles/kg, about 10¹¹ viral particles/kg to about 10¹² viral particles/kg, about 10¹² viral particles/kg to about 10¹³ viral particles/kg, about 10¹³ viral particles/kg to about 10¹⁴ viral particles/kg, or about 10¹⁴ viral particles/kg to about 10¹⁵ viral particles/kg.

A liquid dosage form of an oncolytic vaccinia virus as described herein can comprise, in some embodiments, a viral dose of about 10³ PFU/mL to about 10⁴ PFU/mL, about 10⁴ PFU/mL to about 10⁵ PFU/mL, about 10⁵ PFU/mL to about 10⁶ PFU/mL, about 10⁷ PFU/mL to about 10⁸ PFU/mL, about 10⁹ PFU/mL to about 10¹⁰ PFU/mL, about 10¹⁰ PFU/mL to about 10¹¹ PFU/mL, about 10¹¹ PFU/mL to about 10¹¹ PFU/mL, about 10¹¹ PFU/mL to about 10³ PFU/mL, about 10¹³ PFU/mL to about 10¹⁴ PFU/mL, or about 10¹⁴ PFU/mL to about 10¹ PFU/mL. In some embodiments, a modified oncolytic virus of this disclosure, such as an oncolytic vaccinia virus, can be administered at a dose that can comprise about 2×10³ PFU/mL, 3×10³ PFU/mL, 4×10³ PFU/mL, 5×10³ PFU/mL, 6×10³ PFU/mL, 7×10³ PFU/mL, 8×10³ PFU/mL, 9×10³ PFU/mL, about 10⁴ PFU/mL, about 2×10⁴ PFU/mL, about 3×10⁴ PFU/mL, about 4×10⁴ PFU/mL, about 5×10⁴ PFU/mL, about 6×10⁴ PFU/mL, about 7×10⁴ PFU/mL, about 8×10⁴ PFU/mL, about 9×10⁴ PFU/mL, about 10⁵ PFU/mL, 2×10⁵ PFU/mL, 3×10⁵ PFU/mL, 4×10⁵ PFU/mL, 5×10⁵ PFU/mL, 6×10⁵ PFU/mL, 7×10⁵ PFU/mL, 8×10⁵ PFU/mL, 9×10⁵ PFU/mL, about 10⁶ PFU/mL, about 2×10⁶ PFU/mL, about 3×10⁶ PFU/mL, about 4×10⁶ PFU/mL, about 5×10⁶ PFU/mL, about 6×10⁶ PFU/mL, about 7×10⁶ PFU/mL, about 8×10⁶ PFU/mL, about 9×10⁶ PFU/mL, about 10⁷ PFU/mL, about 2×10⁷ PFU/mL, about 3×10⁷ PFU/mL, about 4×10⁷ PFU/mL, about 5×10⁷ PFU/mL, about 6×10⁷ PFU/mL, about 7×10⁷ PFU/mL, about 8×10⁷ PFU/mL, about 9×10⁷ PFU/mL, about 10⁸ PFU/mL, about 2×10⁸ PFU/mL, about 3×10⁸ PFU/mL, about 4×10⁸ PFU/mL, about 5×10⁸ PFU/mL, about 6×10⁸ PFU/mL, about 7×10⁸ PFU/mL, about 8×10⁸ PFU/mL, about 9×10⁸ PFU/mL, about 10⁹ PFU/mL, about 2×10⁹ PFU/mL, about 3×10⁹ PFU/mL, about 4×10⁹ PFU/mL, about 5×10⁹ PFU/mL, about 6×10⁹ PFU/mL, about 7×10⁹ PFU/mL, about 8×10⁹ PFU/mL, about 9×10⁹ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹¹ PFU/mL, about 2×10¹¹ PFU/mL, about 3×10¹¹ PFU/mL, about 4×10¹¹ PFU/mL, about 5×10¹¹ PFU/mL, about 6×10¹¹ PFU/mL, about 7×10¹¹ PFU/mL, about 8×10¹¹ PFU/mL, about 9×10¹¹ PFU/mL, or about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a modified oncolytic virus of this disclosure, such as an oncolytic vaccinia virus, can be administered at a dose that can comprise 5×10⁹ PFU/mL. In some embodiments, a modified oncolytic virus of this disclosure, such as an oncolytic vaccinia virus, can be administered at a dose that can comprise up to 5×10⁹ PFU/mL.

In some instances, where the modified oncolytic virus is administered by an injection, the dosage can comprise about 10³ viral particles per injection, 10⁵ viral particles per injection, 10⁵ viral particles per injection, 10⁶ viral particles per injection, 10⁷ viral particles per injection, 10⁸ viral particles per injection, 10⁹ viral particles per injection, 10¹⁰ viral particles per injection, 10¹¹ viral particles per injection, 10¹² viral particles per injection, 2×10¹² viral particles per injection, 10¹³ viral particles per injection, 10¹⁴ viral particles per injection, or 10¹⁵ viral particles per injection. In further instances, where the modified oncolytic virus is administered by an injection, the dosage can comprise about 10³ infectious viral particles per injection, 10⁴ infectious viral particles per injection, 10⁵ infectious viral particles per injection, 10⁴ infectious viral particles per injection, 10⁷ infectious viral particles per injection, 10⁸ infectious viral particles per injection, 10⁹ infectious viral particles per injection, 10¹⁰ infectious viral particles per injection, 10¹¹ infectious viral particles per injection, 10¹¹ infectious viral particles per injection, 2×10¹² infectious viral particles per injection, 10¹³ infectious viral particles per injection, 10¹⁴ infectious viral particles per injection, or 10¹⁵ infectious viral particles per injection. In some embodiments, the virus can be administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor.

In some embodiments, a single dose of a pharmaceutical composition described herein can refer to the amount administered to a subject or a tumor over a 1, 2, 5, 10, 15, 20 or 24 hour period. In some embodiments, the dose can be spread over time or by separate injection. In some embodiments, multiple doses (e.g., 2, 3, 4, 5, 6 or more doses) of the pharmaceutical composition described herein can be administered to the subject, for example, where a second treatment can occur within 1, 2, 3, 4, 5, 6, 7 days or weeks of a first treatment. In some embodiments, multiple doses of a pharmaceutical composition described herein can be administered to the subject over a period of 1, 2, 3, 4, 5, 6, 7 or more days or weeks. In some embodiments, the a pharmaceutical composition described herein or the pharmaceutical composition as described herein can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the oncolytic vaccinia virus or the pharmaceutical composition as described herein can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In some embodiments of the methods disclosed herein, the oncolytic vaccinia virus or the pharmaceutical composition can be administered, independently, in an initial dose for a first period of time, an intermediate dose for a second period of time, and a high dose for a third period of time. In some embodiments, the initial dose is lower than the intermediate dose and the intermediate dose is lower than the high dose. In some embodiments, the first, second, and third periods of time are, independently, about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer.

One or more doses of a pharmaceutical composition comprise a treatment cycle. Two or more doses in a treatment cycle may be separated by a dose interval, wherein no pharmaceutical composition is administered. One or more treatment cycles comprise a course of treatment. Two or more treatment cycles in a course of treatment may be separated by a treatment interval, wherein no treatment is provided.

In some embodiments of methods described herein, one or more doses of a composition, cell, fusion protein, oncolytic virus, or vaccinia virus described herein comprise a treatment cycle. In some embodiments, 1, 2, 3, 4, 5, 6, 7, 8, 9, 10, or more doses of a composition, cell, fusion protein, oncolytic virus, or vaccinia virus described herein comprise a treatment cycle. In some embodiments, a dose is administered over about 1 minute, about 1 hour, about 2 hours, about 3 hours, about 4 hours, about 1 day, or more. In some embodiments, all doses in a treatment cycle are about the same in dose amount and duration. In some embodiments, each dose in a treatment cycle is independent of any other doses. In some embodiments, two or more doses in a treatment cycle are separated by a dose interval, wherein no doses are administered. In some embodiments, a dose interval is about 1 minute, about 1 hour, about 2 hours, about 3 hours, about 4 hours, about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, or more. In some embodiments, all dose intervals are about the same. In some embodiments, each dose interval is independent of any other dose interval.

In some embodiments of methods described herein, one or more treatment cycles comprise a course of treatment. In some embodiments, 1, 2, 3, 4, 5, 6, 7, 8, 9, 10 or more treatment cycles comprise a course of treatment. A treatment cycle described herein can be from 1 to 24 hours, from 1 to 6 days, from 1 to 4 weeks, from 1 to 12 months or more in duration. In some embodiments, a treatment cycle is 1 hour, 2 hours, 3 hours, 4 hours, 5 hours 10 hours, 15 hours, 20 hours, 1 day, 2 days, 3 days, 4 days, 5 days, 6 days, 1 week, 2 weeks, 3 weeks, 4 weeks, 1 month, 2 months, 3 months, 4 months 5 months, 6 months 7 months, 8 months, 9 months, 10 months, 11 months, 12 months, or more. In some embodiments, all treatment cycles are about the same. In some embodiments, each treatment cycle is independent of any other treatment cycles. In some embodiments, two or more treatment cycles are separated by a treatment interval during which no treatment is administered. In some embodiments, a treatment interval is from 1 to 6 days, from 1 to 4 weeks, from 1 to 12 months, from 1 to 5 years, or more. In some embodiments, a treatment interval is about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, or more. In some embodiments, all treatment intervals are about the same. In some embodiments, each treatment interval is independent of any other treatment interval.

In some embodiments of methods described herein, one or more courses of treatment with a composition, cell, fusion protein, oncolytic virus, or vaccinia virus described herein comprise a method described herein. In some embodiments, 1, 2, 3, 4, 5, 6, 7, 8 or more courses of treatment comprise a method. In some embodiments, a course of treatments is from 1 to 6 days, from 1 to 4 weeks, from 1 to 12 months, from 1 to 5 years, or more. In some embodiments, a course of treatment is about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, or more. In some embodiments, all courses of treatment are the same. In some embodiments, each course of treatment is independent of any other courses of treatment. In some embodiments, each course of treatment is followed by a course interval, wherein no treatment is administered. In some embodiments, the course interval is from 1 to 6 days, from 1 to 4 weeks, from 1 to 12 months, from 1 to 5 years, or more. In some embodiments, the course interval is about 1 day, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, or more. In some embodiments, the duration of a course interval is dependent on an incidence or recurrence of a disease. In some embodiments, all course intervals about the same. In some embodiments, each course interval is independent of any other course interval.

In some examples, the subject can be put on a reduced carbohydrate diet, e.g., a ketogenic diet prior to, concurrent with, and following administration of the modified oncolytic viruses, such as the oncolytic vaccinia viruses or the pharmaceutical composition comprising the same, as described herein, according to any of the methods of treatment described herein. In some embodiments, the subject is put on a diet that can comprise consuming less than 500 grams of carbohydrates per day, less than 450 grams of carbohydrates per day, less than 450 grams of carbohydrates per day, less than 400 grams of carbohydrates per day, less than 350 grams of carbohydrates per day, less than 300 grams of carbohydrates per day, less than 250 grams of carbohydrates per day, less than 200 grams of carbohydrates per day, less than 150 grams of carbohydrates per day, less than 100 grams of carbohydrates per day, less than 90 grams of carbohydrates per day, less than 80 grams of carbohydrates per day, less than 70 grams of carbohydrates per day, less than 60 grams of carbohydrates per day, less than 50 grams of carbohydrates per day, less than 40 grams of carbohydrates per day, less than 30 grams of carbohydrates per day, less than 20 grams of carbohydrates per day, less or than 10 grams of carbohydrates per day.

An exemplary method for the delivery of a modified oncolytic virus of the present disclosure, such as an oncolytic vaccinia virus as described herein or a pharmaceutical composition comprising the same, to cancer or tumor cells can be via intratumoral injection. However, alternate methods of administration can also be used. The routes of administration can vary with the location and nature of the tumor. In some embodiments, the administration can be a local administration or a systemic administration. In some embodiments, the route of administration is intravenous, regional (e.g., in the proximity of a tumor, particularly with the vasculature or adjacent vasculature of a tumor), intraperitoneal, parenteral, intramuscular, subcutaneous, intraarterial, percutaneous, intrathecal, intratracheal, intravesical, transdermal, intradermal, by inhalation, perfusion, intranasal, intraurethral, intravaginal, by lavage, orally, intradental, rectally, or any combination thereof. An injectable dose of the oncolytic virus can be administered as a bolus injection or as a slow infusion. In some embodiments, the modified oncolytic virus can be administered to the patient from a source implanted in the patient. In some embodiments, administration of the modified oncolytic virus can occur by continuous infusion over a selected period of time. In some instances, an oncolytic vaccinia virus as described herein, or a pharmaceutical composition comprising the same can be administered at a therapeutically effective dose by infusion over a period of about 15 mins, about 30 mins, about 45 mins, about 50 mins, about 55 mins, about 60 minutes, about 75 mins, about 90 mins, about 100 mins, or about 120 mins or longer. The oncolytic virus or the pharmaceutical composition of the present disclosure can be administered as a liquid dosage, wherein the total volume of administration is about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL.

### Pharmaceutical Compositions

Provided herein are pharmaceutical compositions comprising a fusion construct or a nucleic acid encoding for such construct, and a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of pharmaceutically acceptable carriers include buffers, water, emulsions, various types of wetting agents, sterile solutions, preservatives, stabilizers, compaction agents, lubricants, chelators, dispersion enhancers, disintegration agents, and any combination thereof. In some embodiments, a buffer comprises a citrate buffer, a phosphate buffer, an acetate buffer, or any combination thereof. In some embodiments, the emulsion is an oil in water (O/W) emulsion, a water in oil emulsion (W/O), or a multiple emulsion. Additional non-limiting examples of pharmaceutically acceptable carriers can include gels, bioadsorbable polymers, implantation elements comprising the fusion construct or nucleic acid, or any other suitable vehicle, delivery or dispensing means or material. In some embodiments, the bioadsorbable polymer comprises polylactic acid (PLA), polyglycolic acid (PGA), polycaprolactone (PCL), polydioxone (PDO), or any combination thereof. In some embodiments, the gel comprises a protein, a polysaccharide, a cellulose derivative, a synthetic polymer, or any combination thereof. In some embodiments, the protein comprises gelatin, collagen, or any combination thereof. In some embodiments, the polysaccharide comprises pectin, gellum gum, alginic acid, agar, xanthin, cassia tora, tragacanth, sodium or potassium carrageenan, guar gum, or any combination thereof. In some embodiments, the cellulose derivative comprises methylcellulose, hydroxyethyl cellulose, hydroxypropyl cellulose, hydroxypropyl methyl cellulose, or any combination thereof. In some embodiments, the synthetic polymer comprises a carbomer, a polyacrylamide, a poloxamer, a polyvinyl alcohol, a polyethylene or its co-polymers, or any combination thereof. Such carriers can be formulated by conventional methods and can be administered to the subject at an effective amount.

In some embodiments, pharmaceutical compositions described herein are formulated as a water-based solution, an oil-based solution, a dispersion in glycerol, a liquid polyethylene glycol, a solid, an inhalable form, an intranasal form, liposomes, nanoparticles, microparticles, polymers, or any combination thereof. In some embodiments, a pharmaceutical composition as described herein can comprise a stabilizer and a buffer. In some embodiments, a pharmaceutical composition as described herein can comprise a solubilizer, such as sterile water or a buffer.

### Methods of Production

In some embodiments, modified oncolytic viruses of this disclosure are packaged in cell lines. In some embodiments, the modified oncolytic virus can be propagated in suitable host cells, e.g., HeLa cells, 293 cells, or Vero cells, isolated from host cells and stored in conditions that promote stability and integrity of the virus, such that loss of infectivity over time is minimized. In certain exemplary methods, the modified oncolytic viruses are propagated in host cells using cell stacks, roller bottles, or perfusion bioreactors. In some examples, downstream methods for purification of the modified oncolytic viruses can comprise filtration (e.g., depth filtration, tangential flow filtration, or a combination thereof), ultracentrifugation, or chromatographic capture. The modified oncolytic virus can be stored, e.g., by freezing or drying, such as by lyophilization. In some embodiments, prior to administration, the stored modified oncolytic virus can be reconstituted (if dried for storage) and diluted in a pharmaceutically acceptable carrier for administration. In some embodiments, the modified oncolytic virus as described herein, exhibit a higher titer in HeLa cells and 293 cells compared to an otherwise identical virus that does not comprise the modifications in the modified oncolytic virus. In certain instances, a higher titer in HeLa cells and 293 cells is seen in modified oncolytic virus.

### Kits

In embodiments, this disclosure provides for a kit for administering a modified oncolytic virus as described herein. In some embodiments, a kit of this disclosure can include a modified oncolytic virus or a pharmaceutical composition comprising a modified oncolytic virus as described above. In some embodiments, a kit of this disclosure can further include one or more components such as instructions for use, devices and additional reagents, and components, such as tubes, containers and syringes for performing the methods disclosed above. In some embodiments, a kit of this disclosure can further include one or more agents, e.g., at least one of an anti-cancer agent, an immunomodulatory agent, or any combinations thereof, that can be administered in combination with a modified virus.

In some embodiments, a kit of this disclosure can comprise one or more containers comprising a modified virus, disclosed herein. For example, and not by way of limitation, a kit of this disclosure can comprise one or more containers that comprise a modified oncolytic virus of this disclosure.

In some embodiments, a kit of this disclosure can include instructions for use, a device for administering the modified oncolytic virus to a subject, or a device for administering an additional agent or compound to a subject. For example, and not by way of limitation, the instructions can include a description of the modified oncolytic virus and, optionally, other components included in the kit, and methods for administration, including methods for determining the proper state of the subject, the proper dosage amount and the proper administration method for administering the modified virus. Instructions can also include guidance for monitoring the subject over duration of the treatment time.

In some embodiments, a kit of this disclosure can include a device for administering the modified oncolytic virus to a subject. Any of a variety of devices known in the art for administering medications and pharmaceutical compositions can be included in the kits provided herein. For example, and not by way of limitation, such devices include, a hypodermic needle, an intravenous needle, a catheter, a needle-less injection device, an inhaler and a liquid dispenser, such as an eyedropper. In some embodiments, a modified oncolytic virus to be delivered systemically, for example, by intravenous injection, an intratumoral injection, an intraperitoneal injection, can be included in a kit with a hypodermic needle and syringe.

### Exemplary Embodiments

Provided herein are fusion proteins, wherein the fusion protein comprises: a TNF (tumor necrosis factor)-superfamily ligand (TNFSF-L) or functional variant thereof; and a plurality of domains from a collectin family protein, wherein the plurality of domains from the collectin family protein comprises: an oligomerization domain or functional variant thereof; and a neck domain or functional variant thereof, wherein the oligomerization domain or functional variant thereof and the neck domain or functional variant thereof are closer in sequence proximity compared to their location in the collectin family protein. Further provided herein are fusion proteins, wherein the fusion protein comprises the following, in N-terminal to C-terminal order: the oligomerization domain or functional variant thereof; the neck domain or functional variant thereof; optionally, a linker sequence; and the TNFSF-L or functional variant thereof. Further provided herein are fusion proteins, wherein the TNFSF-L is Lymphotoxin alpha, OX40 ligand, CD40 ligand, Fas ligand, CD27 ligand, CD30 ligand, 4-1BBL, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18, Ectodysplasin A. Further provided herein are fusion proteins, wherein the TNFSF-L peptide is CD40 ligand. Further provided herein are fusion proteins, wherein the CD40 ligand comprises at least 85% sequence identity to SEQ ID NO: 1. Further provided herein are fusion proteins, wherein the TNFSF-L peptide is OX40 ligand. Further provided herein are fusion proteins, wherein the OX40 ligand comprises at least 85% sequence identity to SEQ ID NO: 2 or SEQ ID NO: 3. Further provided herein are fusion proteins, wherein the TNFSF-L peptide is 4-1BBL. Further provided herein are fusion proteins, wherein the 4-1BBL comprises at least 85% sequence identity to SEQ ID NO: 4 or SEQ ID NO: 5. Further provided herein are fusion proteins, wherein the TNFSF-L peptide is LIGHT. Further provided herein are fusion proteins, wherein the LIGHT comprises at least 85% sequence identity to SEQ ID NO: 6. Further provided herein are fusion proteins, wherein the collectin family protein is SP-A, SP-D, mannose binding lectin (MBL), conglutinin, CL-43, CL-L1, CL-K1, CL-P1, or CL-46. Further provided herein are fusion proteins, wherein the collectin family protein is SP-D. Further provided herein are fusion proteins, wherein the oligomerization domain comprises at least 85% sequence identity to SEQ ID NO: 9. Further provided herein are fusion proteins, wherein the oligomerization domain and neck domain together comprise at least 85% sequence identity to SEQ ID NO: 11. Further provided herein are fusion proteins, wherein the oligomerization domain and neck domain together comprise SEQ ID NO: 11. Further provided herein are fusion proteins, wherein the fusion protein comprises the linker sequence, and wherein the linker sequence comprises GSG (glycine-serine-glycine) (SEQ ID NO: 12).

Provided herein are fusion proteins, wherein the fusion protein comprises a sequence having at least 85% sequence identity to SEQ ID NOS: 14, 15, 16, 18, or 19.

Provided herein are oncolytic viruses, wherein the oncolytic virus comprises: a TNF (tumor necrosis factor)-superfamily ligand (TNFSF-L) or functional variant thereof fused to an oligomerization domain. Further provided herein are oncolytic viruses, wherein the oncolytic virus is Newcastle disease virus (NDV), Reovirus (RV), Myxoma virus (MYXV), Measles virus (MV), Herpes Simplex virus (HSV), Vaccinia virus (VV), Vesicular Somatitis virus (VSV) or Polio virus (PV). Further provided herein are oncolytic viruses, wherein the TNFSF-L or functional variant thereof is inserted into the viral genome. Further provided herein are oncolytic viruses, wherein the TNFSF-L or functional variant thereof is inserted into a thymidine kinase gene.

Provided herein are vaccinia viruses, wherein the vaccinia virus comprises: a TNF (tumor necrosis factor)-superfamily ligand (TNFSF-L) or functional variant thereof fused to an oligomerization domain. Further provided herein are vaccinia viruses, wherein the vaccinia virus is a modified strain of Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Further provided herein are vaccinia viruses, wherein the TNFSF-L or functional variant thereof is inserted into the viral genome. Further provided herein are vaccinia viruses, wherein the TNFSF-L or functional variant thereof is inserted into a thymidine kinase gene.

Provided herein are oncolytic viruses, wherein the oncolytic virus comprises the fusion protein as described herein.

Provided herein are vaccinia viruses, wherein the vaccinia virus comprises the fusion protein as described herein.

Provided herein are methods for treatment of cancer, comprising: administering a vector comprising a nucleic acid to a subject in an amount sufficient for treatment a cancer, wherein the nucleic acid encodes for the fusion protein as described herein.

Provided herein are methods for treatment of cancer, comprising: administering the oncolytic virus as described herein to a subject in an amount sufficient for treatment of a cancer.

Provided herein are methods for treatment of cancer, comprising: administering the vaccinia virus as described herein to a subject in an amount sufficient for treatment of a cancer.

Provided herein are methods for reduction of tumor cell growth, comprising: administering a vector comprising a nucleic acid to tumor cells in an amount sufficient for reduction of tumor cell growth, wherein the nucleic acid encodes for the fusion protein as described herein.

Provided herein are methods for reduction of tumor cell growth, comprising: administering the oncolytic virus as described herein to tumor cells in an amount sufficient for reduction of tumor cell growth.

Provided herein are methods for reduction of tumor cell growth, comprising: administering the vaccinia virus as described herein to tumor cells in an amount sufficient for reduction of tumor cell growth.

### EXAMPLES

The examples below further illustrate the described embodiments without limiting the scope of this disclosure.

### Example 1: Expression of Native and TNFSF-L Fusion Constructs

The following fusion TNFSF-L constructs were designed. 3sCD40L (murine); 3sOX40L (murine); 3s41BBL (murine); 3sOX40L (human); and 3s41BBL (human). Sequences for these constructs are described in Table 4 and Table 5.

In addition, the following native monomer producing constructs were designed mOX40L (murine, native) and m41BBL (murine, native). Sequences for these constructs are provided in Table 6 (amino acid) and Table 7 (nucleic acid).

TABLE 6

| SEQ ID | | |
|---|---|---|
| NO: | Description | Amino Acid Sequence |
| 28 | mOX40L | MEGEGVQPLDENLENGSRPRFKWKKTLRLVVSGIKGAGMLLCFIYVC |
| | (murine, | LQLSSSPAKDPPIQRLRGAVTRCEDGQLFISSYKNEYQTMEVQNNSV |
| | native) | VIKCDGLYIIYLKGSFFQEVKIDLHFREDHNPISIPMLNDGRRIVFT |
| | | VVASLAFKDKVYLTVNAPDTLCEHLQINDGELIVVQLTPGYCAPEGS |
| | | YHSTVNQVPL* |
| 29 | m4-1BBL | MDQHTLDVEDTADARHPAGTSCPSDAALLRDTGLLADAALLSDTVRP |
| | (murine, | TNAALPTDAAYPAVNVRDREAAWPPALNFCSRHPKLYGLVALVLLLL |
| | native) | IAACVPIFTRTEPRPALTITTSPNLGTRENNADQVTPVSHIGCPNTT |
| | | QQGSPVFAKLLAKNQASLCNTTLNWHSQDGAGSSYLSQGLRYEEDKK |
| | | ELVVDSPGLYYVFLELKLSPTFTNTGHKVQGWVSLVLQAKPQVDDFD |
| | | NLALTVELFPCSMENKLVDRSWSQLLLLKAGHRLSVGLRAYLHGAQD |
| | | AYRDWELSYPNTTSFGLFLVKPDNPWE* |
| 30 | mOX40L | ATGGAAGGGGAAGGGGTTCAACCCCTGGATGAGAATCTGGAAAACGG |
| | (murine, | ATCAAGGCCAAGATTCAAGTGGAAGAAGACGCTAAGGCTGGTGGTCT |
| | native) | CTGGGATCAAGGGAGCAGGGATGCTTCTGTGCTTCATCTATGTCTGC |
| | | CTGCAACTCTCTTCCTCTCCGGCAAAGGACCCTCCAATCCAAAGACT |
| | | CAGAGGAGCAGTTACCAGATGTGAGGATGGGCAACTATTCATCAGCT |
| | | CATACAAGAATGAGTATCAAACTATGGAGGTGCAGAACAATTCGGTT |
| | | GTCATCAAGTGCGATGGGCTTTATATCATCTACCTGAAGGGCTCCTT |
| | | TTTCCAGGAGGTCAAGATTGACCTTCATTTCCGGGAGGATCATAATC |
| | | CCATCTCTATTCCAATGCTGAACGATGGTCGAAGGATTGTCTTCACT |
| | | GTGGTGGCCTCTTTGGCTTTCAAAGATAAAGTTTACCTGACTGTAAA |
| | | TGCTCCTGATACTCTCTGCGAACACCTCCAGATAAATGATGGGGAGC |
| | | TGATTGTTGTCCAGCTAACGCCTGGATACTGTGCTCCTGAAGGATCT |
| | | TACCACAGCACTGTGAACCAAGTACCACTGTGA |
| 31 | m4-1BBL | ATGGACCAGCACACACTTGATGTGGAGGATACCGCGGATGCCAGACA |
| | (murine, | TCCAGCAGGTACTTCGTGCCCCTCGGATGCGGCGCTCCTCAGAGATA |
| | native) | CCGGGCTCCTCGCGGACGCTGCGCTCCTCTCAGATACTGTGCGCCCC |
| | | ACAAATGCCGCGCTCCCCACGGATGCTGCCTACCCTGCGGTTAATGT |
| | | TCGGGATCGCGAGGCCGCGTGGCCGCCTGCACTGAACTTCTGTTCCC |
| | | GCCACCCAAAGCTCTATGGCCTAGTCGCTTTGGTTTTGCTGCTTCTG |
| | | ATCGCCGCCTGTGTTCCTATCTTCACCCGCACCGAGCCTCGGCCAGC |
| | | GCTCACAATCACCACCTCGCCCAACCTGGGTACaCGAGAGAATAATG |
| | | CAGACCAGGTCACCCCTGTTTCCCACATTGGCTGCCCCAACACTACA |
| | | CAACAGGGCTCTCCTGTGTTCGCCAAGCTACTGGCTAAAAACCAAGC |
| | | ATCGTTGTGCAATACAACTCTGAACTGGCACAGCCAAGATGGAGCTG |
| | | GGtctTCATACCTATCTCAAGGTCTGAGGTACGAAGAAGACAAAAAG |
| | | GAGTTGGTGGTAGACAGTCCCGGGCTCTACTACGTATTTTTGGAACT |
| | | GAAGCTCAGTCCAACATTCACAAACACAGGCCACAAGGTGCAGGGCT |
| | | GGGTCTCTCTTGTTTTGCAAGCAAAGCCTCAGGTAGATGACTTTGAC |
| | | AACTTGGCCCTGACAGTGGAACTGTTCCCTTGCTCCATGGAGAACAA |
| | | GTTAGTGGACCGTTCCTGGAGTCAACTGTTGCTCCTGAAGGCTGGCC |
| | | ACCGCCTCAGTGTGGGTCTGAGGGCTTATCTGCATGGAGCCCAGGAT |
| | | GCATACAGAGACTGGGAGCTGTCTTATCCCAACACCACCAGCTTTGG |
| | | ACTCTTTCTTGTGAAACCCGACAACCCATGGGAATGA |

### Table 7.

Expression of TNFSF-L native monomer or variant fusion constructions from oncolytic viruses was performed. Briefly, HeLa cells were infected at an MOI of 5.0 with viruses expressing monomers or scaffold-TNFSF-L fusions (for OX40L or 41BBL) or with control virus not expressing TNFSF-L, or virus expressing scaffold-TNFSF-L fusion (for CD40L). Media was collected at 24 hours and run on commercial ELISA kit as per manufacturer's instructions (samples were undiluted or diluted as indicated). Samples were diluted 1:100, binding recorded, and amounts summarized in the charts account for the dilution factor.

Referring to FIG. 2, uninfected HeLa cells did not show detectible levels of mCD40L. Likewise, HeLa cells infected with oncolytic vaccinia virus having A52R and TK deletions did not show detectible levels of mCD40L. HeLa cells infected with oncolytic vaccinia virus having TK deletion and the fusion construct 3sCD40L (murine) having an oligomerization domain and inserted in the TK gene shows much higher level expression, about 400 ng/mL.

Referring to FIG. 3, uninfected HeLa cells did not show detectible levels of mOX40L. Likewise, HeLa cells infected with oncolytic vaccinia virus having A52R and TK deletions did not show detectible levels of mOX40L. HeLa cells infected with oncolytic vaccinia virus having TK deletion and native mOX40L inserted in the TK gene showed slightly increased mOX40L expression, with about 4500 μg/mL detected. HeLa cells infected with oncolytic vaccinia virus having TK deletion and fusion construct 3sOX40L (murine) comprising the oligomerization domain inserted in the TK gene had much higher levels of mOX40L expression, with about 265,000 μg/mL mOX40L detected.

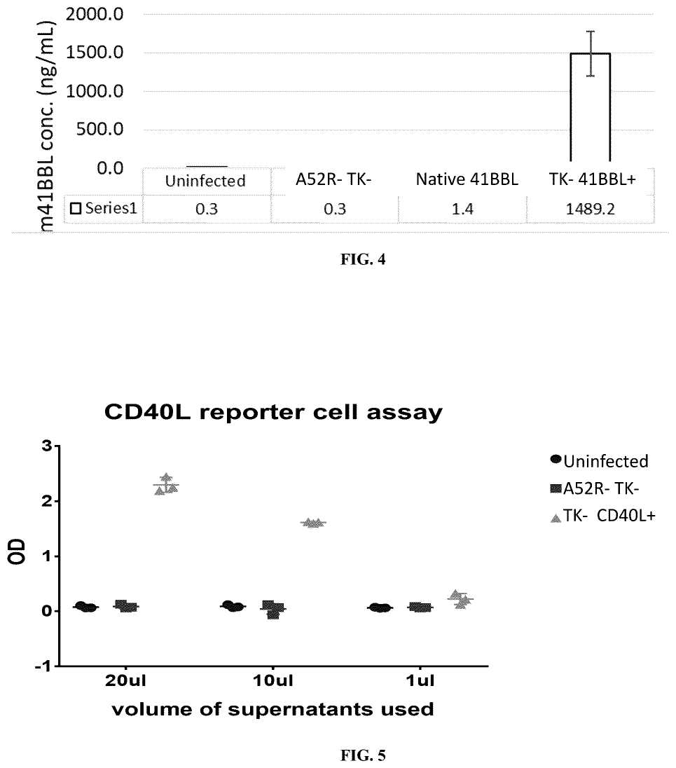

Referring to FIG. 4, uninfected HeLa cells did not show detectible levels of m41BBL. HeLa cells infected with oncolytic vaccinia virus having A52R and TK deletions likewise did not show detectible levels of m41BBL. HeLa cells infected with oncolytic vaccinia virus having TK deletion and native m41BBL inserted in the TK gene showed minimal expression, with 1.4 ng/mL detected. HeLa cells infected with oncolytic vaccinia virus having TK deletion and fusion construct 3s41BBL (murine) comprising the oligomerization domain inserted in the TK gene had much higher levels of 41BBL expression, with about 1500 ng/mL 41BBL detected.

### Example 2: Expression of Native and Scaffold Structures

Functional effects of modified oncolytic vaccinia virus expressing a fusion construct of TNFSF-L having an oligomerization domain was assayed. A SEAP reporter assay of CD40L activity was used, where the amount of SEAP production is correlative of CD40L functional activity. Briefly, HeLa cells were infected with oncolytic vaccinia virus (no CD40L) or oncolytic vaccinia virus expressing fusion construct 3sCD40L comprising: [oligomerization domain]-[neck domain]-[mCD40L] at an MOI of 1. The supernatant of infected and uninfected cells was collected after 24 hours. To a 96 well plate 20 ul, 10 ul or 1 ul of the supernatants was added, in triplicates to wells with 20 ul of media. Recombinant CD40L at 10 ng/ml concentration was used as the positive control. 180 ul of the reporter cell line was added to the wells to arrive at about 50,000 cells per well. Cells were incubated overnight in a C02 incubator. The next day 1 ml Quanti-Blue solution at was added to 1 ml of QB buffer and 98 ml of sterile water, and the prepared reagent was dispensed 180 ul/well to a 96 well plate. 20 ul of the supernatant from the experimental plate was added to the plate with the Quanti blue solution. Following incubation at 37° C. for 3 hours, the relative level of SEAP was determined by measuring optical density (O.D.) using a plate reader/spectrophotometer at 625 nM. Results are provided below in Table 8 and visualized in FIG. 5.

TABLE 8

| | | | Vaccinia Virus |
|---|---|---|---|
| | | | with |
| | | | oligomerization |
| | | Vaccinia Virus | domain |
| | | with No transgene | fused to |
| | Mock | (A52R-, TK-) | CD40L (TK-) |
| 20 ul | 0.075 | 0.087 | 2.297 |
| 10 ul | 0.088 | 0.039 | 1.617 |
| 1 ul | 0.063 | 0.07 | 0.223 |
| Control: | 0.29765 | | |
| Recombinant CD40L | (not | | |
| (10 ng/mL) | shown) | | |

Unexpectedly, these results from both Examples 1 and 2 show over 1000× more functionally active TNFSF-L being produced in the extracellular environment. In particular, the monomer sequence did not produce functionally active trimer, even in cell culture, and the fusion to the scaffold domains resulted in a significant increase in production.

### Example 3: Analysis of In Vivo Activity of s3CD40L

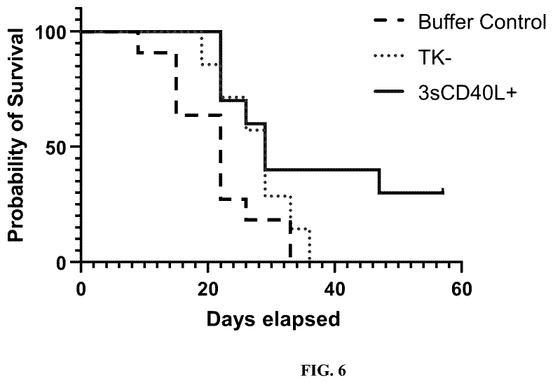

The activity in mice of the TNFSF-L fusion construct 3sCD40L (murine) as described in Example 1 was measured. BALB/c mice were implanted subcutaneously with cells derived from a renal cortical adenocarcinoma in Balb/cCr mice (RENCA). Tumors were allowed to grow to 50-100 mm³ and treated with 1×10⁷ PFU of virus expressing the 3sCD40L fusion construct, having a TK deletion (TK-), or a buffer control. Treatments were delivered intratumorally (IT) in a single dose. Tumor growth was followed and mice were taken off study once tumors measured greater than 1000 mm³. The overall survival in mice with tumors measuring up to 1000 mm³ is shown in FIG. 6. The data indicates mice treated with the TNFSF-L fusion showed a higher probability of survival compared to those treated with TK-virus or buffer control.

### Example 4: Functional Activity of S3 OX40L

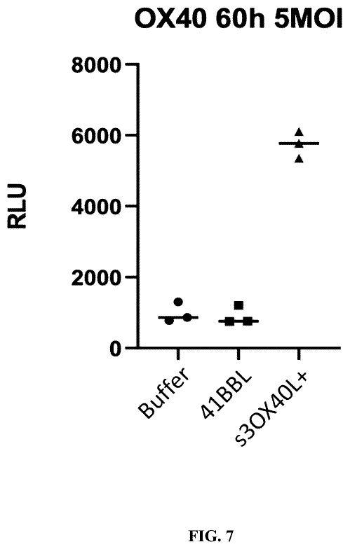

The in vitro activity of the TNFSF-L fusion construct 3sOX40L (human), as described in Example 1, was measured. HeLa cells were plated in 6-well plates at 1×10⁶ cells per well. Cells were inoculated at an MOI of 5.0 with virus expressing the 3sOX40L fusion construct, expressing the m41BBL as described in Example 1 as a negative control, or a buffer control. Cells were incubated with virus at 37° C., 5% CO₂ for 3 hours. Media was removed from wells and cells were washed with DMEM. DMEM+10% FBS was added and plates were incubated for 60 hours at 37° C., 5% CO₂. Supernatants were harvested at 60 hours post-medium replacement. Luciferase expression in infected cells was measured using luciferase detection and expressed in relative light units (RLU). FIG. 7 shows higher expression in the virus having the s3OX40L fusion construct.

### Example 5: Functional Activity of 3s41BBL

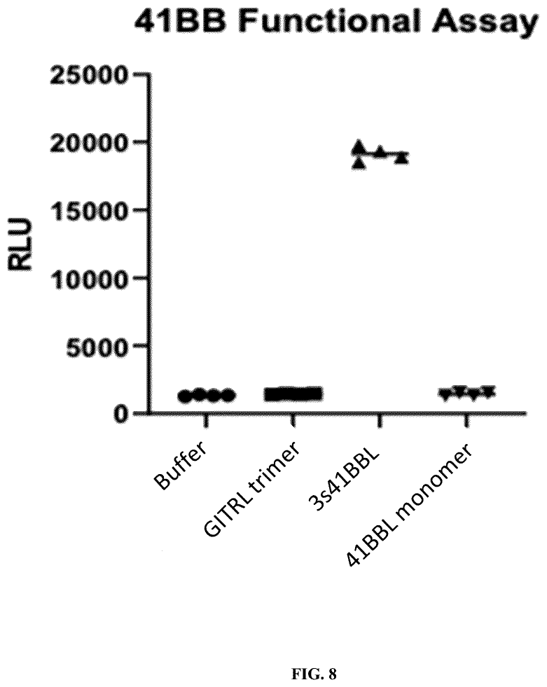

The in vitro activity of the TNFSF-L fusion construct 3s41BBL (human sequence), as described in Example 1, was measured. HeLa cells were plated in 12-well plates at 4×10⁵ cells per well. Cells were infected at an MOI of 5.0 with virus expressing the 3s41BBL fusion construct, expressing 41BBL monomer, expressing an unrelated TNFSF-L, trimeric GITRL, or buffer control. Cells were incubated with virus at 37° C., 5% CO₂ for 48 hours. Supernatants were harvested at 48 hours post-infection. Luciferase expression in infected cells was measured using luciferase detection and expressing in relative light units (RLU). FIG. 8 shows only virus-expressed fusion construct 3s41BBL is active, viral expressed 41BBL monomer is not active.

### Example 6: IL-2 Secretion after Stimulation with Mouse Sequence S3 OX40L and S3 41BBL

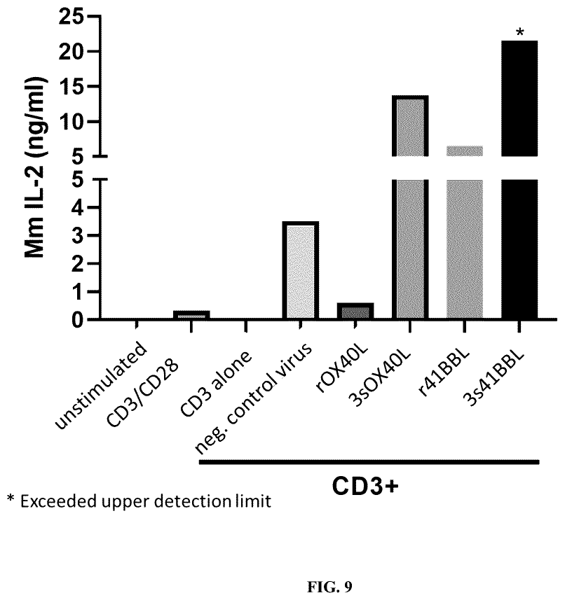

The change in IL-2 secretion in cells infected with virus comprising TNFSF-L fusion constructs was measured. CD8 T-cells from mouse spleen were purified using EasySep™ Mouse CD8+ T Cell Isolation Kit (#19853, STEMCELL Technologies, Inc., Vancouver, Canada). Cells were labelled with carboxyfluorescein succinimidyl ester (CFSE). Cells were then either unstimulated, stimulated with CD3 alone, or stimulated with CD3 and CD28, recombinant 41BBL trimer, recombinant OX40L trimer, virus expressing 3s41BBL fusion construct, virus expressing 3sOX40L fusion construct, or virus negative control. Cells were incubated for 3 days. IL-2 secretion in supernatants was analyzed using a mouse IL-2 ELISA kit. Resulting detected IL-2 levels are shown in FIG. 9. The graph shows virally expressed TNFSF-L fusion constructs 3s41BBL and 3sOX40L are functionally active in that they induce IL-2 expression.

### Example 7: Tumor Growth Inhibition in Mice Using 3S41BBL and 3S OX40L Scaffold Constructs

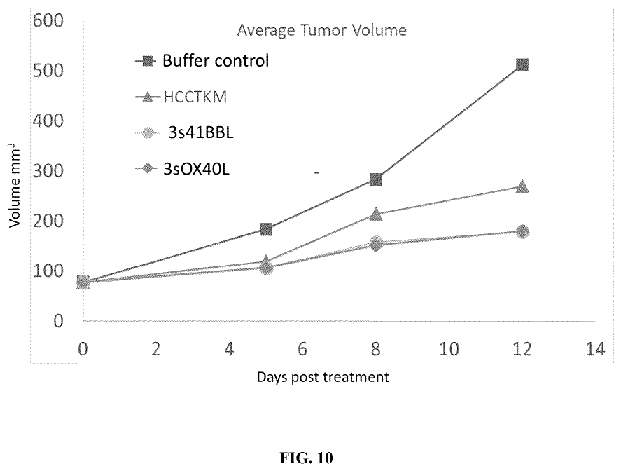

To demonstrate in vivo Tumor Growth Inhibition (TGI), BALB/c mice previously induced with subcutaneous RENCA tumors as in Example 3, were treated with a single IT dose of buffer control, or 1×10⁷ PFU of control vaccinia virus with only a Thymidine Kinase deletion (HCCTKM), virus expressing 3s41BBL or virus expressing 3sOX40L. Preliminary results indicate, as shown in FIG. 10, that to 12 days post treatment, average tumor volume in the mice receiving virus expressing either 3s41BBL or 3sOX40L was lower than in tumors treated with buffer alone or HCCTKM.

### Example 8: Tumor Growth Inhibition in Mice Using GITRL Scaffold Constructs

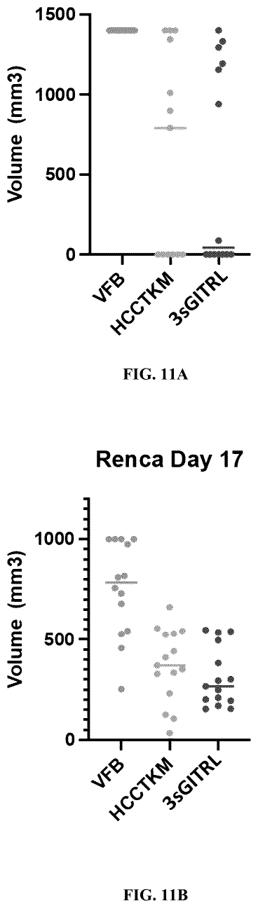

To demonstrate in vivo Tumor Growth Inhibition (TGI), BALB/c mice previously induced with subcutaneous LLC or RENCA tumors as in Example 3, were treated with a single IT dose of buffer control (VFB), 1×10⁷ PFU of control vaccinia virus with only a Thymidine Kinase deletion (HCCTKM), or virus expressing 3sGITRL (. LLC tumors were measured after 30 days, as shown in FIG. 11A. RENCA tumors were measured after 17 days, as shown in FIG. 11B. Results indicate average tumor volume in the mice receiving virus expressing 3sGITRL was lower than in tumors treated with buffer alone or control HCCTKM.

The foregoing description and accompanying drawings set forth a number of representative embodiments at the present time. Various modifications, additions, and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. An oncolytic virus, wherein the oncolytic virus comprises:
an exogenous nucleic acid encoding for a fusion protein, wherein the fusion protein comprises a TNF-superfamily ligand (TNFSF-L) or functional variant thereof, and an oligomerization domain from a collectin family protein.

2. The oncolytic virus of claim 1, wherein the fusion protein further comprises a neck domain or functional variant thereof from a collectin family protein, and wherein the oligomerization domain and the neck domain are closer in sequence proximity compared to their location in the collectin family protein.

3. The oncolytic virus of claim 1, wherein the oncolytic virus comprises a viral genome, and wherein the nucleic acid encoding the TNFSF-L or functional variant thereof is inserted into the viral genome.

4. The oncolytic virus of claim 3, wherein the nucleic acid encoding the TNFSF-L or functional variant thereof is inserted into a thymidine kinase gene of the viral genome.

5. The oncolytic virus of claim 2, wherein the fusion protein comprises, in N-terminal to C-terminal order:
the oligomerization domain or functional variant thereof;
the neck domain or functional variant thereof;
optionally, a linker sequence; and
the TNFSF-L or functional variant thereof.

6. The oncolytic virus of claim 1, wherein the TNFSF-L comprises a Lymphotoxin alpha, OX40 ligand, CD40 ligand, Fas ligand, CD27 ligand, CD30 ligand, 4-1BBL, TNF-related apoptosis-inducing ligand (TRAIL), Receptor activator of nuclear factor kappa-B ligand (RANKL), TNF-related weak inducer of apoptosis, A proliferation-inducing ligand (APRIL), B-cell activating factor (BAFF), LIGHT, Vascular endothelial growth inhibitor (VEGI), TNF superfamily member 18 ligand (GITRL), Ectodysplasin A, or any combination thereof.

7. The oncolytic virus of claim 6, wherein the TNFSF-L is the CD40 ligand and wherein the CD40 1 ligand comprises a sequence comprising at least 85% sequence identity to SEQ ID NO: 1.

8. The oncolytic virus of claim 6, wherein the TNFSF-L is the OX40 ligand and wherein the OX40 ligand comprises a sequence comprising at least 85% sequence identity to SEQ ID NO: 2 or SEQ ID NO: 3.

9. The oncolytic virus of claim 6, wherein the TNFSF-L is a 4-1BBL and wherein the 4-1BBL comprises a sequence comprising at least 85% sequence identity to SEQ ID NO: 4 or SEQ ID NO: 5.

10. The oncolytic virus of claim 6, wherein the TNFSF-L is a LIGHT and wherein the LIGHT comprises a sequence comprising at least 85% sequence identity to SEQ ID NO: 6.

11. The oncolytic virus of claim 6, wherein the TNFSF-L is a TNF superfamily member 18 ligand (GITRL) and wherein the GITRL comprises a sequence comprising at least 85% sequence identity to SEQ ID NO: 7 or SEQ ID NO: 8.

12. The oncolytic virus of claim Error! Reference source not found., wherein the collectin family protein is SP-A, SP-D, mannose binding lectin (MBL), conglutinin, CL-43, CL-L1, CL-K1, CL-P1, or CL-46.

13. The oncolytic virus of claim Error! Reference source not found., wherein the collectin family protein is SP-D.

14. The oncolytic virus of claim 2, wherein the oligomerization and neck domains combined comprise a sequence comprising at least 85% sequence identity to SEQ ID NO: 10.

15. The oncolytic virus of claim 1, wherein the exogenous nucleic acid comprises an RNA.

16. The oncolytic virus of claim 1, wherein the exogenous nucleic acid comprises a DNA.

17. The oncolytic virus of claim 1, wherein the oncolytic virus is Newcastle disease virus (NDV), Reovirus (RV), Myxoma virus (MYXV), Measles virus (MV), Herpes Simplex virus (HSV), Vaccinia virus (VV), Vesicular Somatitis virus (VSV), Polio virus (PV), Sendai virus, Flavivirus, Lentivirus, a pox virus, a retrovirus, an adeno-associated virus, or an adenovirus.

18. The oncolytic virus of claim 17, wherein the oncolytic virus is the vaccinia virus.

19. The oncolytic virus of claim 18, wherein the vaccinia virus is a modified strain of Western Reserve Vaccinia virus (ATCC VR-1354), Copenhagen strain, Vaccinia virus Ankara (ATCC VR-1508), Vaccinia virus Ankara (ATCC VR-1566), recombinant vaccinia virus Ankara (MVA), NYVAC strain, Vaccinia virus strain Wyeth (ATCC VR-1536), Vaccinia virus Wyeth (ATCC VR-325), Wyeth (NYCBOH) strain, Tian Tan strain, Lister strain, USSR strain, and Evans strain.

20. The oncolytic virus of claim 1, further comprising a neck domain or functional variant thereof, wherein the nucleic acid encoding for the oligomerization domain and the neck domain comprises SEQ ID NO: 11.

21. A method for treatment of cancer, comprising:
administering a pharmaceutical composition to a subject in an amount sufficient for treatment of a cancer, wherein the pharmaceutical composition comprises:
the oncolytic virus of claim 1.


SEQUENCE LISTING (31 sequences)

SEQ ID NO: 1 - protein, 214 aa, Mus sp.
HRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 2 - protein, 126 aa, Homo sapiens
RIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - protein, 144 aa, Mus sp.
AKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 4 - protein, 177 aa, Mus sp.
SHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 5 - protein, 166 aa, Homo sapiens
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYTYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 7 - protein, 132 aa, Mus sp.
TSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGIILMPDLPFIS

SEQ ID NO: 8 - protein, 132 aa, Homo sapiens; identical to SEQ ID NO: 7

SEQ ID NO: 9 - protein, 52 aa, synthetic construct
  REGION 1..52: note = Description of Artificial Sequence: Syntheticpolypeptide
MLLFLLSALVLLTQPLGYLEAEMKTYSHRTMPSACTLVMCSSVESGLPGRDG

SEQ ID NO: 10 - protein, 83 aa, synthetic construct
  REGION 1..83: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVE

SEQ ID NO: 11 - DNA, 249 bp, synthetic construct
  misc_feature 1..249: note = Description of Artificial Sequence: Syntheticpolynucleotide
  source 1..249: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggatctgatgttgcttctctgaggcagcaggttgaggccttacagggacaagtacagcacctccaggctgctttctctcagtataagaaagtggag

SEQ ID NO: 12 - skipped

SEQ ID NO: 13 - protein, 54 aa, synthetic construct
  REGION 1..54: note = Description of Artificial Sequence: Syntheticpolypeptide
  VARIANT 1..18: note = This region may encompass 0-6 Gly Ser Ser repeating units
  VARIANT 19..36: note = This region may encompass 0-6 Ser Ser Gly repeating units
  VARIANT 37..54: note = This region may encompass 0-6 Gly Ser Gly repeating units
GSSGSSGSSGSSGSSGSSSSGSSGSSGSSGSSGSSGGSGGSGGSGGSGGSGGSG

SEQ ID NO: 14 - protein, 300 aa, synthetic construct
  REGION 1..300: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVEGSGHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 15 - protein, 232 aa, synthetic construct
  REGION 1..232: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVESSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 16 - protein, 261 aa, synthetic construct
  REGION 1..261: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVESSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 17 - protein, 218 aa, synthetic construct
  REGION 1..218: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVEGSGTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGIILMPDLPFIS

SEQ ID NO: 18 - protein, 212 aa, synthetic construct
  REGION 1..212: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVEGSGRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 19 - protein, 252 aa, synthetic construct
  REGION 1..252: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVEGSGQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 20 - protein, 211 aa, synthetic construct
  REGION 1..211: note = Description of Artificial Sequence: Syntheticpolypeptide
MLLFLLSALVLLTQPLGYLEAEMKTYSHRTMPSACTLVMCSSVESGLPGRDGSDVASLRQQVEALQGQVQHLQAAFSQYKKVEGSGTAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 21 - DNA, 903 bp, synthetic construct
  misc_feature 1..903: note = Description of Artificial Sequence: Syntheticpolynucleotide
  source 1..903: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggatctgatgttgcttctctgaggcagcaggttgaggccttacagggacaagtacagcacctccaggctgctttctctcagtataagaaagtggagggatccggtcacaggcgtctggacaaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga

SEQ ID NO: 22 - DNA, 699 bp, synthetic construct
  misc_feature 1..699: note = Description of Artificial Sequence: Syntheticpolynucleotide
  source 1..699: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagcggcctgcctggtcgcgatggatctgatgttgcttctctgaggcagcaggttgaggccttacagggacaagtacagcacctccaggctgctttctctcagtataagaaagtagaatcatcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgcgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgtga

SEQ ID NO: 23 - DNA, 786 bp, synthetic construct
  misc_feature 1..786: note = Description of Artificial Sequence: Syntheticpolynucleotide
  source 1..786: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggatctgatgttgcttctctgaggcagcaggttgaggccttacagggacaagtacagcacctccaggctgctttctctcagtataagaaagtagaatcatcccacattggctgccccaacactacacaacagggctctcctgtgttcgccaagctactggctaaaaaccaagcatcgttgtgcaatacaactctgaactggcacagccaagatggagctgggtcttcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatga

SEQ ID NO: 24 - DNA, 657 bp, synthetic construct
  misc_feature 1..657: note = Description of Artificial Sequence: Syntheticpolynucleotide
  source 1..657: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggatctgatgttgcttctctgaggcagcaggttgaggccttacagggacaagtacagcacctccaggctgctttctctcagtataagaaagtggagggatccggtacttcactcaagccaactgccatcgagtcctgcatggttaagtttgaactatcatcctcaaaatggcacatgacatctcccaaacctcactgtgtgaatacgacatctgatgggaagctgaagatactgcagagtggcacatatttaatctacggccaagtgattcctgtggataagaaatacataaaagacaatgcccccttcgtagtacagatatataaaaagaatgatgtcctacaaactctaatgaatgattttcaaatcttgcctataggaggggtttatgaactgcatgctggagataacatatatctgaagttcaactctaaagaccatattcagaaaactaacacatactgggggatcatcttaatgcctgatctaccattcatctcttag

SEQ ID NO: 25 - DNA, 639 bp, synthetic construct
  misc_feature 1..639: note = Description of Artificial Sequence: Syntheticpolynucleotide
  source 1..639: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggatctgatgttgcttctctgaggcagcaggttgaggccttacagggacaagtacagcacctccaggctgctttctctcagtataagaaagtggagggatccggtcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 26 - DNA, 715 bp, synthetic construct
  misc_feature 1..715: note = Description of Artificial Sequence: Syntheticpolynucleotide
  source 1..715: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggatctgatgttgcttctctgaggcagcaggttgaggccttacagggacaagtacagcacctccaggctgctttctctcagtataagaaagtgtgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 27 - DNA, 636 bp, synthetic construct
  misc_feature 1..636: note = Description of Artificial Sequence: Syntheticpolynucleotide
  source 1..636: mol_type = other DNA
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggatctgatgttgcttctctgaggcagcaggttgaggccttacagggacaagtacagcacctccaggctgctttctctcagtataagaaagtggagggatccggtactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 28 - protein, 198 aa, synthetic construct
  REGION 1..198: note = Description of Artificial Sequence: Syntheticpolypeptide
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 29 - protein, 309 aa, synthetic construct
  REGION 1..309: note = Description of Artificial Sequence: Syntheticpolypeptide
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 30 - DNA, 597 bp, synthetic construct
  misc_feature 1..597: note = Description of Artificial Sequence: Syntheticpolynucleotide
  source 1..597: mol_type = other DNA
atggaaggggaaggggttcaacccctggatgagaatctggaaaacggatcaaggccaagattcaagtggaagaagacgctaaggctggtggtctctgggatcaagggagcagggatgcttctgtgcttcatctatgtctgcctgcaactctcttcctctccggcaaaggaccctccaatccaaagactcagaggagcagttaccagatgtgaggatgggcaactattcatcagctcatacaagaatgagtatcaaactatggaggtgcagaacaattcggttgtcatcaagtgcgatgggctttatatcatctacctgaagggctcctttttccaggaggtcaagattgaccttcatttccgggaggatcataatcccatctctattccaatgctgaacgatggtcgaaggattgtcttcactgtggtggcctctttggctttcaaagataaagtttacctgactgtaaatgctcctgatactctctgcgaacacctccagataaatgatggggagctgattgttgtccagctaacgcctggatactgtgctcctgaaggatcttaccacagcactgtgaaccaagtaccactgtga

SEQ ID NO: 31 - DNA, 930 bp, synthetic construct
  misc_feature 1..930: note = Description of Artificial Sequence: Syntheticpolynucleotide
  source 1..930: mol_type = other DNA
atggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacacgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggtcttcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatga